(19) United States
(12) Patent Application Publication
(10) Pub. No.: US 2024/0170099 A1
(43) Pub. Date: May 23, 2024

(54) METHYLATION-BASED AGE PREDICTION AS FEATURE FOR CANCER CLASSIFICATION

(71) Applicant: GRAIL, LLC, Menlo Park, CA (US)

(72) Inventors: Onur Sakarya, Redwood City, CA (US); Oliver Claude Venn, San Francisco, CA (US)

(21) Appl. No.: 18/361,023

(22) Filed: Jul. 28, 2023

Related U.S. Application Data
(60) Provisional application No. 63/392,980, filed on Jul. 28, 2022.

(51) Int. Cl. G16B 40/20; G16B 30/00; G16H 50/50

(52) U.S. Cl. CPC G16B 40/20; G16B 30/00; G16H 50/50

(57) ABSTRACT

Methods and systems are disclosed for covariate prediction from methylation features. A system identifies a feature set of genomic regions by training one or more regressions to evaluate a covariance score of a genomic region. The system may select the feature set with the highest indicativeness scores and may consider other selection criteria. The system trains an age prediction model using training samples with reported chronological age label(s). The system can further utilize the chronological age prediction to predict a likelihood of cancer in a test sample. To do so, the system may compare the predicted covariate value and/or label to the reported value and/or label. In one embodiment, the system may utilize an age residual threshold to determine whether there is a strong likelihood of presence of cancer. In other embodiments, the system may utilize the predicted chronological age value as a feature to a cancer classifier.

### CROSS-REFERENCE TO RELATED APPLICATIONS

This application is claims benefit to U.S. Provisional Application No. 63/392,980, filed Jul. 28, 2022, which is incorporated by reference in its entirety.

### BACKGROUND

### Field of Art

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS) or targeted methylation sequencing) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. As part of cancer classification, there remains a need to understand the effect covariate variables (or, more generally, variables that indicate cancer or non-cancer) may have on the human genome. Moreover, there remains a need to be able to distinguish variables that may indicate cancer and/or some other biological attribute such as age or sex.

The present disclosure is directed to addressing the above-referenced challenge. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

In some aspects, the techniques described herein relate to a method for detecting the presence or absence of cancer in a test sample. The method includes obtaining a plurality of training samples, each training sample: including a plurality of nucleic acid fragments, where each of the plurality of nucleic acid fragments has a genomic location overlapping at least one genomic region of a plurality of genomic regions, and is labelled with a chronological age of an individual from whom the training sample is derived. The method includes sequencing the plurality of nucleic acid fragments for each training sample to identify a methylation pattern for each nucleic acid fragment. For each genomic region of a plurality of genomic regions, the method identifies nucleic acid fragments from the plurality having genomic locations overlapping the genomic region, and calculates, for the genomic region, an indicativeness score representing a correlation between chronological age and methylation patterns, and calculated based on chronological ages of individuals from whom the identified nucleic acid fragments are derived and methylation patterns of identified nucleic acid fragments. The method includes generating a feature set including one or more genomic regions of the plurality of genomic regions, the one or more genomic regions in the feature set having indicativeness scores above a threshold. The method includes training a machine-learned age-prediction model to determine a predicted chronological age of a tested individual from whom a test sample is derived, the training based on methylation patterns of nucleic acid fragments in the plurality of training samples overlapping the one or more genomic regions in the feature set.

In some aspects, the method further includes training a linear regression for each genomic region of the feature set based on the methylation patterns of the nucleic acid fragments overlapping each genomic region from training samples of the plurality labelled as non-cancer. The method obtains a plurality of additional training samples. Each additional training sample: includes a plurality of additional nucleic acid fragments having additional genomic locations overlapping at least one genomic region of the plurality of genomic regions, is labelled with a chronological age of an individual from whom the additional training sample was derived, and is labelled as non-cancer or cancer based on a previous determination of cancer presence in the additional training sample. The method includes sequencing the plurality of additional nucleic acid fragments to identify a methylation pattern for each additional nucleic acid fragment. For each genomic region of the plurality, the method applies the linear regression to methylation patterns of nucleic acid fragments of the plurality of additional training samples to determine a predicted chronological age of the individual from whom the additional training sample was derived, calculates age residuals for each additional training sample as a difference between its predicted chronological age and its labelled chronological age, and compares age residuals of the additional training samples labelled as cancer to age residuals of the additional training samples labelled as non-cancer. The method includes generating a reduced feature set from the feature set based on the comparison of age residuals, wherein the reduced feature set includes a lesser number of genomic regions than the feature set, and the reduced feature set is used to train the machine-learned age-prediction model.

In some aspects, the method further includes obtaining a test sample, the test sample including a plurality of additional nucleic acid fragments and labelled with a chronological age of a test subject from whom the test sample is derived. The method includes sequencing the plurality of additional nucleic acid fragments for the test sample to identify methylation patterns for the additional nucleic acid fragments of the plurality. The method includes applying the trained age-prediction model to determine a predicted chronological age of the test subject from whom the test sample was derived based on methylation patterns of the additional nucleic acid fragments overlapping the one or more genomic regions in the feature set, and calculating an age residual as a difference between the labelled chronological age and the predicted chronological age of the test subject. The method includes determining that the test sample has a strong likelihood for presence of cancer in response to determining that the age residual is above a residual threshold.

In some aspects, the method determines the residual threshold by applying the trained age-prediction model to a second plurality of training samples identified as non-cancer to determine a predicted age for each of the second plurality of training samples and calculating an age residual for each of the second plurality of training samples by comparing the predicted age to a labelled chronological age of the second plurality of training samples. The determination includes identifying the residual threshold based on the calculated age residuals for the second plurality of training samples, wherein at least a majority of the calculated age residuals for the second plurality of training samples satisfy the residual threshold.

In some aspects, the method further includes, in response to determining that the test sample has the strong likelihood for presence of cancer: filtering the methylation patterns of the plurality of additional nucleic acid fragments with p-value filtering to identify a set of anomalous methylation patterns, generating a feature vector for the test sample based on the age residual and the set of anomalous methylation patterns, and determining a cancer prediction for the test sample by inputting the feature vector into a trained cancer classifier.

In some aspects, the cancer prediction used in the method is a binary prediction between presence and absence of cancer or another disease state.

In some aspects, the cancer prediction used in the method is a multiclass prediction between a plurality of cancer types.

In some aspects, the cancer prediction used in the method is a multiclass prediction between a plurality of disease states.

In some aspects, the method further includes determining a presence of cancer in the test sample using a secondary machine-learned cancer classifier, the secondary cancer classifier configured to receive as input the predicted chronological age of the subject and methylation patterns of the plurality of additional nucleic acid fragments and output a prediction of the presence of cancer in the test sample.

In some aspects, the secondary machine-learned cancer classifier is further configured to receive as input clinical information and genetic background of the subject and output the prediction of the presence of cancer in the test sample.

In some aspects, the indicativeness score is a Pearson's correlation, or a covariance score.

In some aspects, the indicativeness score is determined by training a linear regression to regress chronological age from methylation density of non-cancer training samples. In this case the methylation density is calculated as a percentage of nucleic acid fragments having genomic locations which overlap a particular genomic region having a methylated state in that particular genomic region.

In some aspects, the machine-learned age-prediction model includes a multivariate regression. The multivariate regression may be penalized based on a number of the one or more genomic regions in the feature set. The machine-learned age-prediction model may receive as input a methylation density corresponding to each of the genomic regions in the feature set.

In some aspects, a number of the one or more genomic regions in the feature set is selected from a range of 5-10,000. Further, sequencing the nucleic acid fragments includes whole genome bisulfate sequencing (WGBS), and/or sequencing the nucleic acid fragments includes targeted sequencing.

In some aspects, the each training sample of the plurality is previously determined to not include a cancer presence, or each training sample of the plurality is previously determined to include a cancer presence. Further, each training sample of the plurality is previously determined to include a cancer presence or a cancer non-presence. In cases where each training sample of the plurality is labelled as having cancer presence or not having cancer presence, the label based on a previous determination of a cancer state for the training sample.

In some aspects, the techniques described herein relate to a method for training a classifier. The method includes obtaining a plurality of training samples, each training sample: including a plurality of nucleic acid fragments, each of the plurality of nucleic acid fragments having a genomic location overlapping at least one genomic region of a plurality of genomic regions, and labelled with a characteristic of an individual from whom the training sample is derived. The method includes sequencing the plurality of nucleic acid fragments for each training sample to identify a methylation pattern for each nucleic acid fragment. For each genomic region of a plurality of genomic regions, the method identifies nucleic acid fragments from the plurality having genomic locations overlapping the genomic region, and calculates, for the genomic region, an indicativeness score representing a correlation between characteristic and methylation patterns, and calculated based on characteristics of individuals from whom the identified nucleic acid fragments are derived and methylation patterns of identified nucleic acid fragments. The method includes generating a feature set including one or more genomic regions of the plurality of genomic regions, the one or more genomic regions in the feature set having indicativeness scores above a threshold. The method includes training a machine-learned characteristics-prediction model to determine a predicted characteristic of a tested individual from whom a test sample is derived, the training based on methylation patterns of nucleic acid fragments in the plurality of training samples overlapping the one or more genomic regions in the feature set.

In some aspects, the characteristic is a biological sex of the individual, and the characteristic is either biological male or biological female. Alternatively or additionally, the characteristic is a smoking status of the individual, and the characteristic is either smoking or non-smoking.

In some aspects, the machine-learned characteristics-prediction model includes a logistic regression implementing a sigmoid function.

In some aspects, the method further includes obtaining a test sample, the test sample including a plurality of additional nucleic acid fragments and labelled with a label indicating a characteristic of the test sample. The method further includes sequencing the additional plurality of nucleic acid fragments for the test sample to identify a test methylation pattern for each additional nucleic acid fragment, and applying the trained machine-learned characteristics-prediction model to predict the characteristic for the test sample based on the methylation patterns of the additional nucleic acid fragments overlapping the feature set of genomic regions. The method, if the predicted label is different than the label of the test sample, includes flagging the test sample as contaminated and withholding the test sample from further analysis.

In some aspects, if the predicted characteristic matches the labelled characteristic, the method further includes filtering the methylation patterns of the additional nucleic acid fragments of the test sample with p-value filtering to identify a set of anomalous methylation patterns; generating a feature vector for the test sample based on the set of anomalous methylation patterns, and determining a cancer prediction for the test sample by inputting the feature vector into a trained cancer classifier.

In some aspects, the cancer prediction is a binary prediction between presence and absence of cancer or another disease state. The cancer prediction may be a multiclass prediction between a plurality of cancer types or a plurality of disease states.

In another aspect, a system comprising a hardware processor and a non-transitory computer-readable storage medium storing instructions that, when executed by the hardware processor, cause the hardware processor to perform the methods disclosed herein. Similarly a non-transitory computer readable storage medium storing instructions that, when executed by one or more processors, cause the processors to perform the methods disclosed herein.

### BRIEF DESCRIPTION OF DRAWINGS

FIG. 1 is an exemplary flowchart describing an overall workflow of cancer classification of a sample, according to one or more embodiments.

FIG. 2A is an exemplary flowchart describing a process of sequencing a fragment of cell-free (cf) DNA to obtain a methylation state vector, according to one or more embodiments.

FIG. 2B is an exemplary illustration of the process of FIG. 2A of sequencing a fragment of cell-free (cf) DNA to obtain a methylation state vector, according to one or more embodiments.

FIG. 3A illustrates methylation features that can be derived from a single CpG site as a genomic region, according to one or more embodiments.

FIG. 3B illustrates methylation features that can be derived from multiple CpG sites as a genomic region, according to one or more embodiments.

FIG. 4A is an exemplary flowchart describing a process of training a chronological age prediction model, according to one or more embodiments.

FIG. 4B illustrates deployment of a chronological age prediction model, according to one or more embodiments.

FIG. 5A is an exemplary flowchart describing a process of generating a control group data structure for determining anomalously methylated fragments, according to one or more embodiments.

FIG. 5B is an exemplary flowchart describing a process of determining a fragment to be anomalously methylated based on the control group data structure, according to one or more embodiments.

FIG. 6A is an exemplary flowchart describing a process of training a cancer classifier, according to one or more embodiments.

FIG. 6B illustrates an example generation of feature vectors used for training the cancer classifier, according to one or more embodiments.

FIG. 7A illustrates an exemplary flowchart of devices for sequencing nucleic acid samples according to one or more embodiments.

FIG. 7B is an exemplary block diagram of an analytics system, according to one or more embodiments.

FIG. 8 illustrates genomic regions associated with age, in accordance with one or more example implementations.

FIG. 9A illustrates one process of identifying a feature set of genomic regions informative of the covariate of age, in accordance with one or more example implementations.

FIG. 9B illustrates a graph of results of age prediction on a non-cancer holdout cohort, in accordance with example implementations.

FIG. 9C illustrates a graph of results of age prediction on a cancer cohort, in accordance with example implementations.

FIG. 10A illustrates another process of identifying a feature set of genomic regions informative of the covariate of age, in accordance with one or more example implementations.

FIG. 10B illustrates a graph of results of age prediction on a non-cancer holdout cohort, in accordance with example implementations.

FIG. 10C illustrates a graph of results of age prediction on a cancer cohort, in accordance with example implementations.

FIG. 11 illustrates the spread of the test cohorts over stages of cancer, in accordance with example implementations.

FIG. 12A shows a top series of graphs representing test samples predicted by the cancer classifier to be negative results, in accordance with some example implementations.

FIG. 12B shows a bottom series of graphs representing test samples predicted by the cancer classifier to be positive results, in accordance with some example implementations.

FIG. 13 illustrates one genomic region showing age deceleration of age across cancer types, in accordance with example implementations.

FIG. 14A shows results for a first genomic region that appears to be consistently age accelerating in hematological cancer types, with less consistent age acceleration in other cancer non-hematological cancer types, in accordance with example implementations.

FIG. 14B shows results for a second genomic region that appears to be consistently age decelerating in hematological cancer types, with little to no significant age deceleration in other cancer non-hematological cancer types, in accordance with example implementations.

FIG. 15A illustrates identification of a feature set of genomic regions for predicting biological sex, in accordance with one or more example implementations.

FIG. 15B illustrates results from a trained biological sex prediction model, in accordance with example implementations.

FIG. 16A illustrates identification of a feature set of genomic regions for predicting smoking status, in accordance with one or more example implementations.

FIG. 16B illustrates results from a trained smoking status sex prediction model, in accordance with example implementations.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing their friends and family to the disease.

Recently, early cancer detection technology has progressed towards analyzing genetic fragments (e.g., DNA) in a person's, for example, blood to determine if any of those genetic fragments originate from cancer cells. These new techniques allow doctors to identify a cancer presence in a patient that may not be detectable otherwise. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers—that is, only being identifiable once it has grown to a sufficient size and has become identifiable with some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, they system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include DNA sample quality, sample contamination, fragmentation bias, and accurate indexing. Remedying these problems would yield better genetic data for cancer detection. Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. The created genetic datasets are typically on the order of terabytes, and effectively and efficiently analyzing that amount of data is both procedurally and computationally demanding. For instance, analyzing NGS sequencing involves several baseline processing steps such as, e.g., aligning reads to one another, aligning and mapping reads to a reference genome, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing.

Finally, and perhaps most importantly, accurate identification of anomalous DNA from NGS data to identify a cancer presence is also difficult. To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state of the art techniques and models are further discussed.

One of the problems in creating and appropriately applying a cancer detection model is, as described above, the vast amount of sequencing data to which the model may be applied. Within that sequencing data is a vast array of “cancer signal” and “separate signal,” where cancer signal indicates sequencing data that is indicative of cancer presence or cancer non-presence in a test subject and separate signal indicates sequencing data that is indicative of, e.g., an additional biological or clinical characteristic of the test subject (e.g., age, sex, smoker, etc.). To compound this issue, some of the separate signal may appear to share similar characteristics to the cancer signal, or some of the cancer signal may appear to share similar characteristics to the separate signal. Still further, some of the sequencing data may be both cancer signal and separate signal. For example, the methylation patterns associated with increased age may appear similar to the methylation patterns for increased age, or, in a similar view, a specific methylation pattern may be indicative of both cancer and increased age.

This interplay of signal(s) in the sequencing data is troublesome because it may lead to error in cancer determinations. For instance, a cancer classifier that is inappropriately trained may determine a person has subject signals based on analysis of their cfDNA because methylation of some of the cfDNA appears “chronologically old and cancerous,” when, in fact, the subject is merely chronologically old. As such, great care should be taken in selecting sequencing data for training a cancer classifier when the sequencing data can indicate several closely related biological processes or clinical characteristics. Any methods that improve cancer determination in these situations represent an improvement to the field of early cancer detection.

Additionally, carefully curating a collection of data which accurately indicates cancer and/or separate biological and/or clinical characteristics alleviates the complexity and corresponding computational expense of executing a machine-learned model to indicate or predicted a presence of call cancer (i.e., “call” cancer) in a sample. In effect, by carefully selecting genomic sites for processing by the model that are “strongly” associated with cancer and/or a biological or clinical characteristic, the computational load on a model will be reduced yielding higher speed and efficiency, which represents an improvement to the field of cancer determination. For example, consider an example machine-learned model trained to identify cancer based on methylation described above. In this instance, however, each indicative feature is also associated with a “strength” of cancer indication for that site, or a “strength” of chronological age indication for that site. That is, abnormal methylation at the first site may strongly indicate cancer, while abnormal methylation at a second site may strongly, or weakly indicate chronological age, etc. In this case, having the machine-learned model process genomic sites that are merely “weakly” indicative of cancer introduces computational expense without providing a corresponding benefit to the accuracy or specificity in cancer determination.

To provide a quantitative example, applying the machine-learned model to 100 weakly indicative sites may not provide as much benefit as applying the machine-learned model to 1 strongly indicative site. As such, selecting the appropriate sites for feature sets for processing by a machine-learned model to identify cancer presence and/or biological or clinical characteristics greatly reduces processing cost without greatly reducing the accuracy or specificity of the model. In effect, it may lessen the analytical load from, e.g., millions of genomic sites to tens of thousands of genomic sites without sacrificing model accuracy. More succinctly, reducing the analytical load from millions of genomic sites to tens of thousands of sites, reduces the processing load and processing time by several orders of magnitude. This reduction enables faster cancer detections and more importantly, frees up computer resources for other models and classifications (e.g., processing on additional samples), improves the performance of the computer implementing the model, reduces the monetary cost of such systems, and improves the fields of public health, medicine, diagnostics, treatment, etc. by detecting cancer earlier than even possible by conventional methods.

Another problem associated with the vast amounts of data generated by NGS sequencing is appropriately training a machine-learned model to identify cancer within the large amount of data. For instance, a machine-learned model may be trained to identify cancer by comparing a feature vector to genomic data. The “features” in the feature vector, as set forth below, may be any genomic site with a sufficient depth of abnormally methylated genomic locations that correspond to cancer presence. When building feature vectors across an entire genome, this can lead to, typically, tens of thousands of features, and, as laid out above, some of those features may be more indicative of cancer presence than others. With this context, selecting which features and corresponding genomic data to use in training a machine learned model is difficult. The machine-learned model should be trained and configured to accurately identify cancer presence, but the resulting model should not be overly expensive computationally. In other words, appropriately selecting data and features for training a machine-learned model improves early cancer-detection.

### I.A. Overview of Cancer Classification Workflow

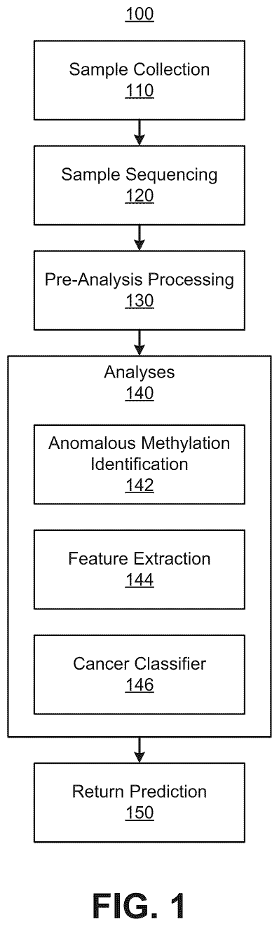

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

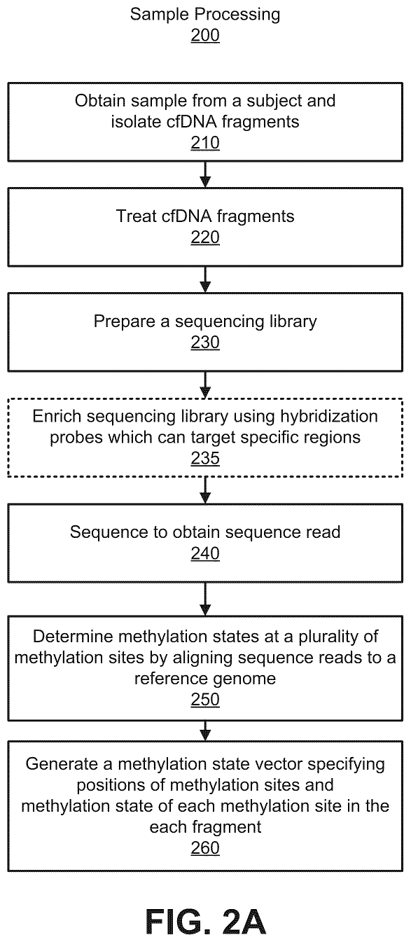

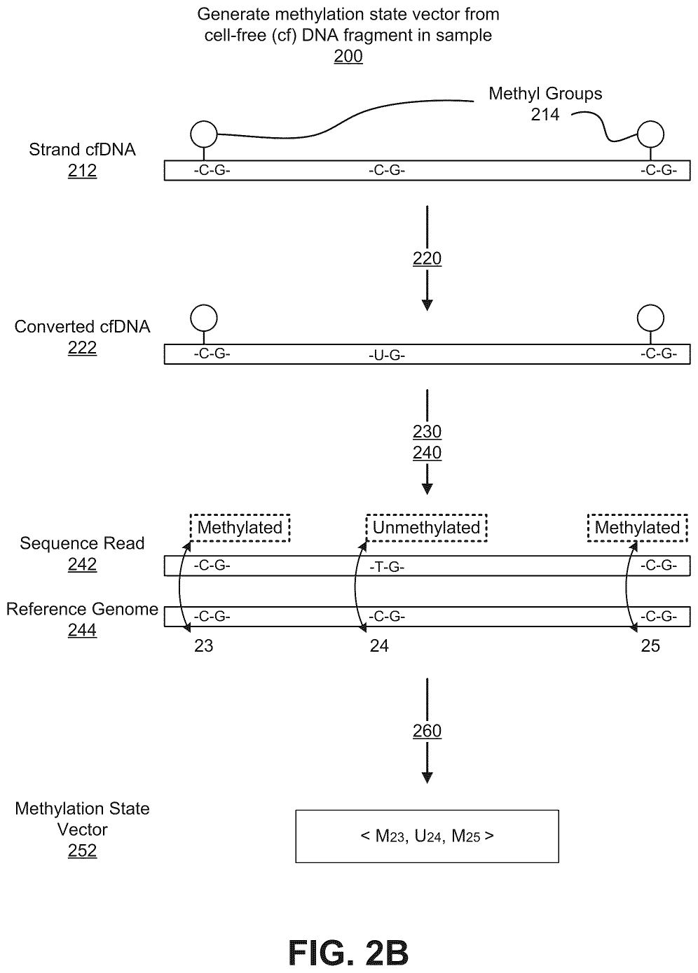

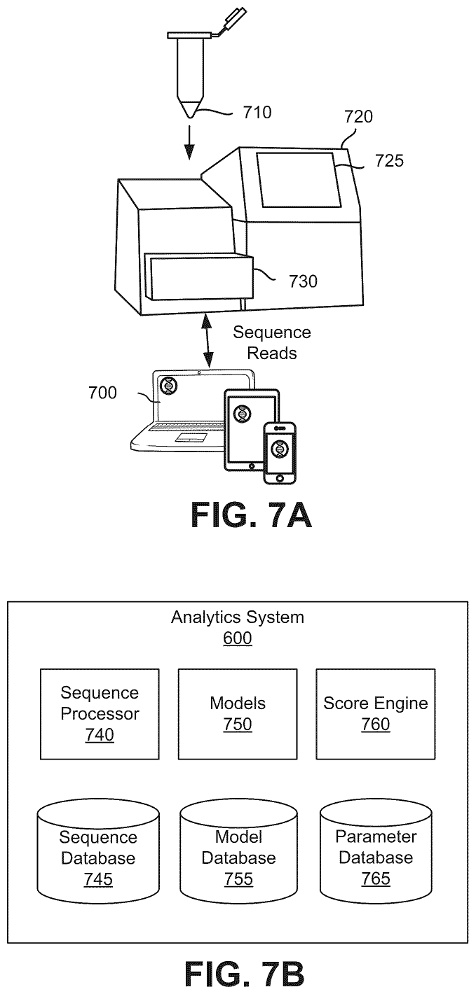

A sequencing device performs sample sequencing 120. A lab clinician may perform one or more processing steps to the sample in preparation of sequencing. Once prepared, the clinician loads the sample in the sequencing device. An example of devices utilizes in sequencing is further described in conjunction with FIGS. 7A & 7B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In context of DNA methylation, bisulfite sequencing (e.g., further described in FIGS. 2A & 2B) can determine methylations status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 7B. Pre-analysis processing 130 may include, but not limited to, de-duplication of sequence reads, determining metrics relating to coverage, determining whether the sample is contaminated, removal of contaminated fragments, calling sequencing error, etc.

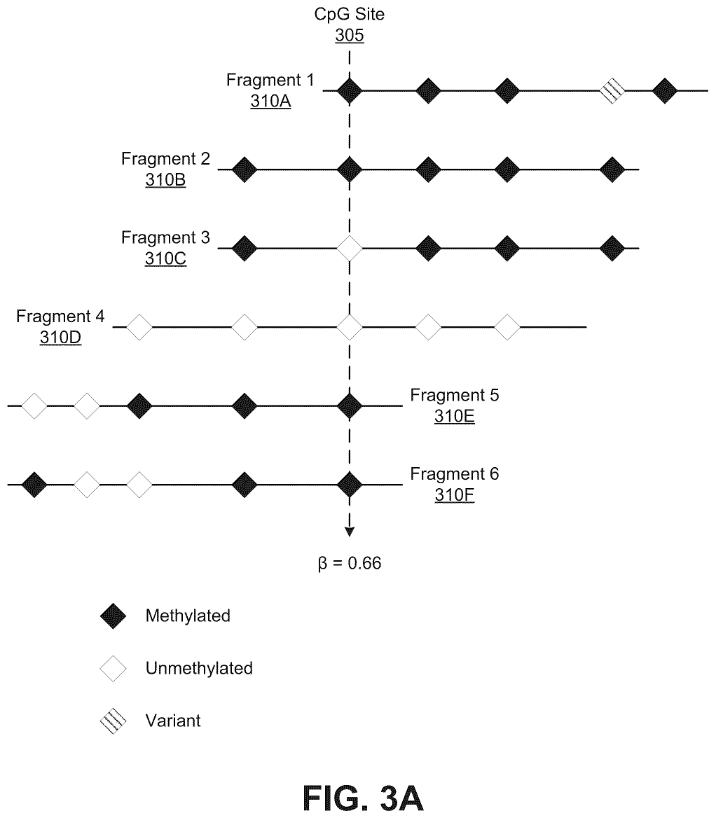

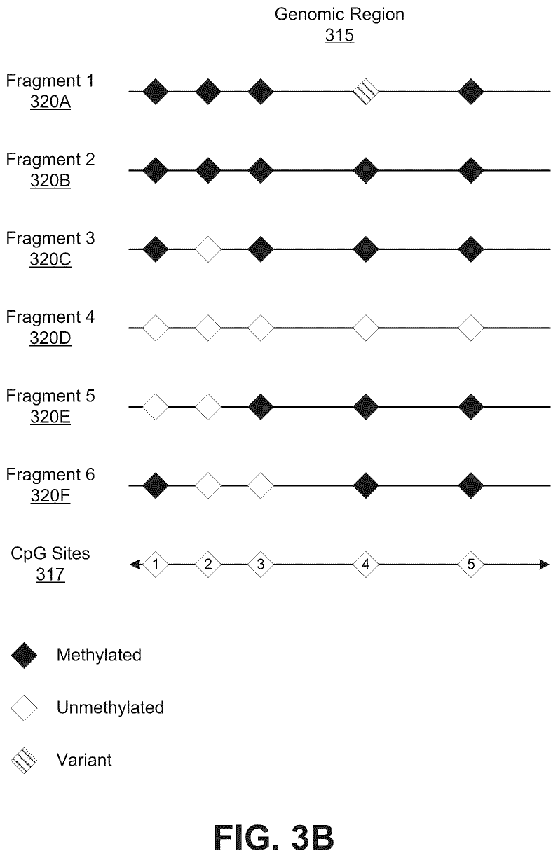

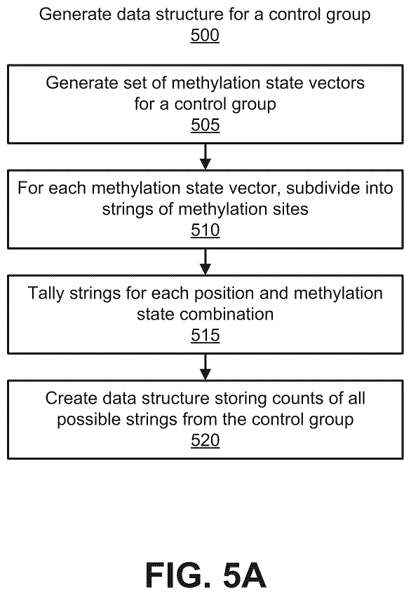

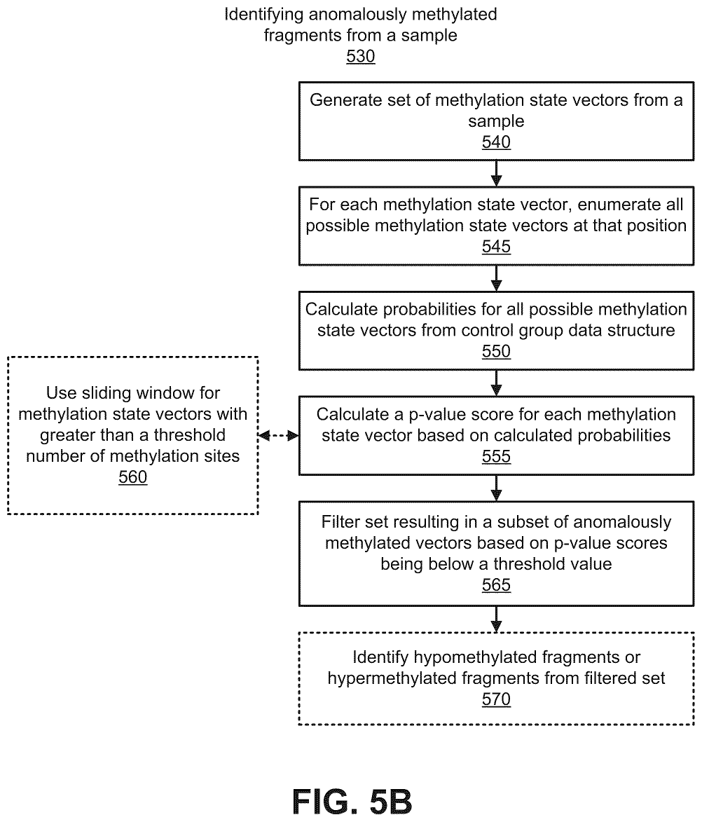

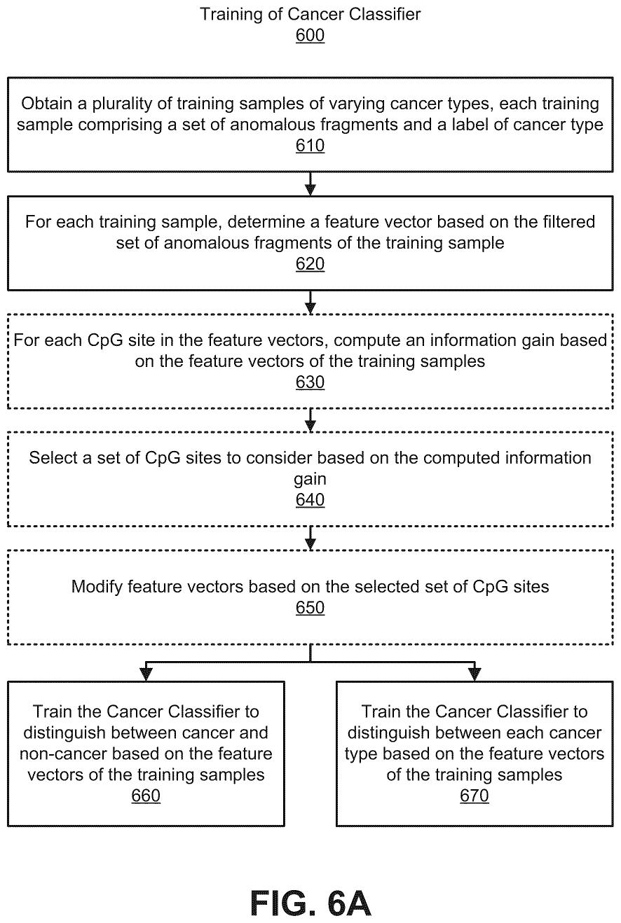

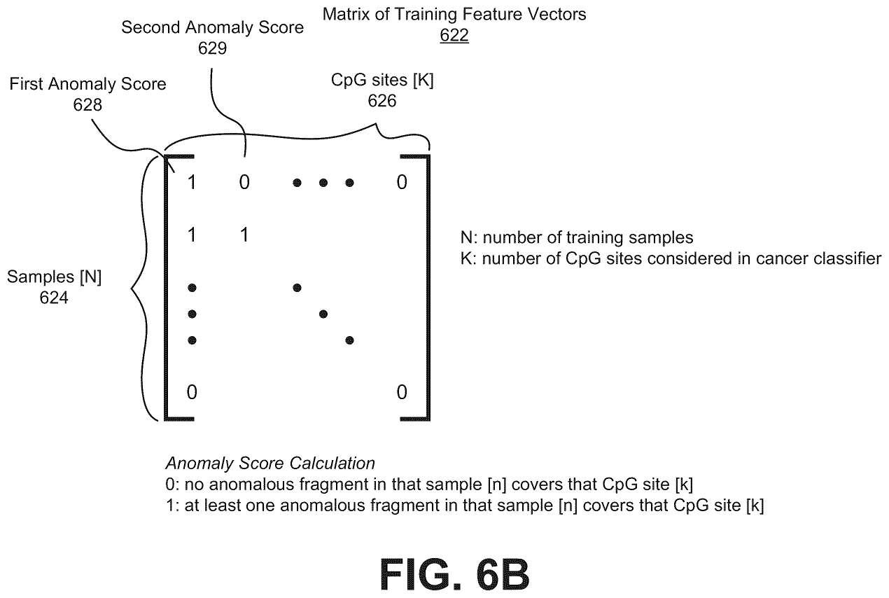

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In context of methylation, analyses 140 may include anomalous methylation identification 142 (e.g., further described in FIGS. 5A & 5B), feature extraction 144 (e.g., further described in FIGS. 3A, 3B, 4A, 4B, 5A, and 5B), and applying a cancer classifier 146 to determine a cancer prediction (e.g., further described in FIGS. 6A & 6B). In one or more embodiments of feature extraction, the analytics system may utilize one or more age prediction models to generate one or more age covariate residuals as features to cancer classification. The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for. The value may indicate a likelihood of a particular cancer state, e.g., a likelihood of cancer, and/or a likelihood of a particular cancer type.

The analytics system returns the prediction 150 to the healthcare provider. The healthcare provider may establish or adjust a treatment plan based on the cancer prediction. Optimization of treatment is further described in Section IV.C. Treatment.

### I.B. Overview of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of anomalously methylated cfDNA fragments. First off, determining a DNA fragment to be anomalously methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “cell free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Anomalous cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

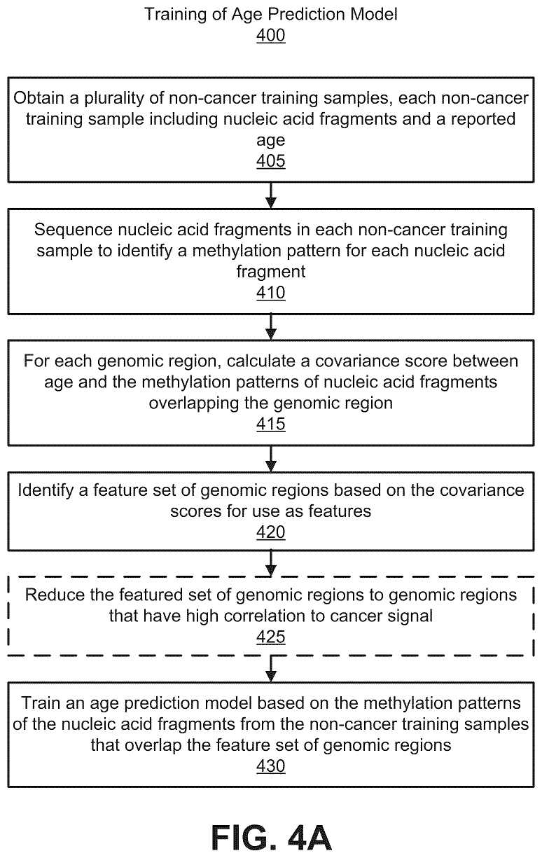

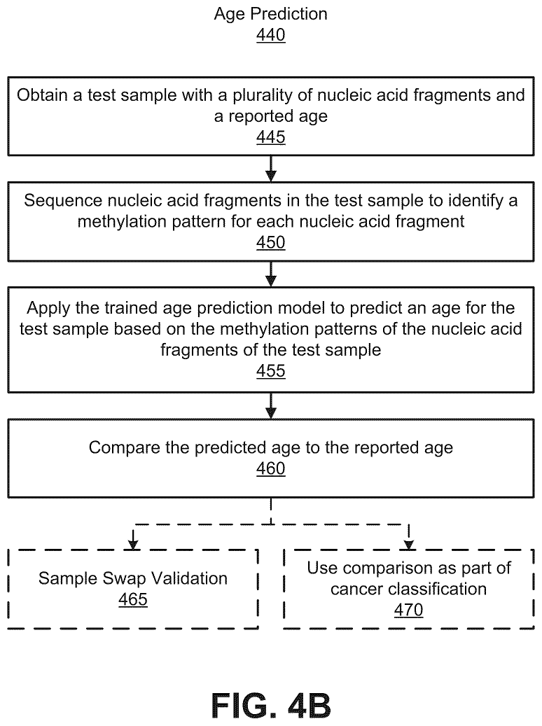

FIG. 7A is an exemplary flowchart of devices for sequencing nucleic acid samples according to one or more embodiments. This illustrative flowchart includes devices such as a sequencer 720 and an analytics system 700. The sequencer 720 and the analytics system 700 may work in tandem to perform one or more steps in the processes 300 of FIG. 3A, 400 of FIG. 4A, 420 of FIG. 4B, and other process described herein.

In various embodiments, the sequencer 720 receives an enriched nucleic acid sample 710. As shown in FIG. 7A, the sequencer 720 can include a graphical user interface 725 that enables user interactions with particular tasks (e.g., initiate sequencing or terminate sequencing) as well as one more loading stations 730 for loading a sequencing cartridge including the enriched fragment samples and/or for loading necessary buffers for performing the sequencing assays. Therefore, once a user of the sequencer 720 has provided the necessary reagents and sequencing cartridge to the loading station 730 of the sequencer 720, the user can initiate sequencing by interacting with the graphical user interface 725 of the sequencer 720. Once initiated, the sequencer 720 performs the sequencing and outputs the sequence reads of the enriched fragments from the nucleic acid sample 710.

In some embodiments, the sequencer 720 is communicatively coupled with the analytics system 700. The analytics system 700 includes some number of computing devices used for processing the sequence reads for various applications such as assessing methylation status at one or more CpG sites, variant calling or quality control. The sequencer 720 may provide the sequence reads in a BAM file format to the analytics system 700. The analytics system 700 can be communicatively coupled to the sequencer 720 through a wireless, wired, or a combination of wireless and wired communication technologies. Generally, the analytics system 700 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 700 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 7B, FIG. 7B is a block diagram of an analytics system 700 for processing DNA samples according to one embodiment. The analytics system implements one or more computing devices for use in analyzing DNA samples. The analytics system 700 includes a sequence processor 740, sequence database 745, model database 755, models 750, parameter database 765, and score engine 760. In some embodiments, the analytics system 700 performs some or all of the processes described throughout this disclosure.

The sequence processor 740 generates methylation state vectors for fragments from a sample. At each CpG site on a fragment, the sequence processor 740 generates a methylation state vector for each fragment specifying a location of the fragment in the reference genome, a number of CpG sites in the fragment, and the methylation state of each CpG site in the fragment whether methylated, unmethylated, or indeterminate via the process 200 of FIG. 2A. The sequence processor 740 may store methylation state vectors for fragments in the sequence database 745. Data in the sequence database 745 may be organized such that the methylation state vectors from a sample are associated to one another.

Further, multiple different models 750 may be stored in the model database 755 or retrieved for use with test samples. In one example, a model is a trained cancer classifier for determining a cancer prediction for a test sample using a feature vector derived from anomalous fragments. The training and use of the cancer classifier will be further discussed in conjunction with Section III. Cancer Classifier for Determining Cancer. The analytics system 700 may train the one or more models 750 and store various trained parameters in the parameter database 765. The analytics system 700 stores the models 750 along with functions in the model database 755.

During inference, the score engine 760 uses the one or more models 750 to return outputs. The score engine 760 accesses the models 750 in the model database 755 along with trained parameters from the parameter database 765. According to each model, the score engine receives an appropriate input for the model and calculates an output based on the received input, the parameters, and a function of each model relating the input and the output. In some use cases, the score engine 760 further calculates metrics correlating to a confidence in the calculated outputs from the model. In other use cases, the score engine 760 calculates other intermediary values for use in the model.

### II. Sample Sequencing & Processing

### II.A. Generating Methylation State Vectors for DNA Fragments

FIG. 2A is an exemplary flowchart describing a process 200 of sequencing a fragment of cfDNA to obtain a methylation state vector, according to one or more embodiments. In order to analyze DNA methylation, an analytics system first obtains 210 a sample from an individual comprising a plurality of cfDNA molecules. In additional embodiments, the process 200 may be applied to sequence other types of DNA molecules. The process 200 is an embodiment of sample sequencing 120 of FIG. 1.

From the sample, the analytics system can isolate 210 each cfDNA molecule. The cfDNA molecules can be treated 220 to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library can be prepared 230. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 235 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfate) for conversion of unmethylated cytosines to uracils. During enrichment, hybridization probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 240 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

From the sequence reads, the analytics system determines 250 a location and methylation state for each CpG site based on alignment to a reference genome. The analytics system generates 260 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as 1). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 2B is an exemplary illustration of the process 200 of FIG. 2A of sequencing a cfDNA molecule to obtain a methylation state vector, according to one or more embodiments. As an example, the analytics system receives a cfDNA molecule 212 that, in this example, contains three CpG sites. As shown, the first and third CpG sites of the cfDNA molecule 212 are methylated 214. During the treatment step 220, the cfDNA molecule 212 is converted to generate a converted cfDNA molecule 222. During the treatment 220, the second CpG site which was unmethylated has its cytosine converted to uracil. However, the first and third CpG sites were not converted.

After conversion, a sequencing library 230 is prepared and sequenced 240 to generate a sequence read 242. The analytics system aligns 250 the sequence read 242 to a reference genome 244. The reference genome 244 provides the context as to what position in a human genome the fragment cfDNA originates from. In this simplified example, the analytics system aligns 250 the sequence read 242 such that the three CpG sites correlate to CpG sites 23, 24, and 25 (arbitrary reference identifiers used for convenience of description). The analytics system can thus generate information both on methylation status of all CpG sites on the cfDNA molecule 212 and the position in the human genome that the CpG sites map to. As shown, the CpG sites on sequence read 242 which are methylated are read as cytosines. In this example, the cytosines appear in the sequence read 242 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 260 a methylation state vector 252 for the fragment cfDNA 212. In this example, the resulting methylation state vector 252 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing.

For further details regarding methylation sequencing (e.g., WGBS and/or targeted methylation sequencing), see, e.g., U.S. patent application Ser. No. 16/352,602, entitled “Methylation Fragment Anomaly Detection,” filed Mar. 13, 2019, and U.S. patent application Ser. No. 16/719,902, entitled “Systems and Methods for Estimating Cell Source Fractions Using Methylation Information,” filed Dec. 18, 2019, each of which is hereby incorporated by reference. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments across each corresponding plurality of nucleic acid methylation fragments can comprise 1000 or more nucleic acid methylation fragments, 5000 or more nucleic acid methylation fragments, 10,000 or more nucleic acid methylation fragments, 20,000 or more nucleic acid methylation fragments, or 30,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 480 nucleotides.

Further details regarding methods for sequencing nucleic acids and methylation sequencing data are disclosed in U.S. patent application Ser. No. 17/191,914, titled “Systems and Methods for Cancer Condition Determination Using Autoencoders,” filed Mar. 4, 2021, which is hereby incorporated herein by reference in its entirety.

### III. Cancer Classifier for Determining Cancer

Cancer classification can involve extracting genetic features and applying one or more models to the extracted features to determine a cancer prediction. The extracted features can include a feature vector generated for a test sample. Cancer classification for the test sample can involve determining a cancer prediction based on the feature vector. The cancer prediction may comprise a label and/or a value. The label may be binary, indicating a presence or absence of cancer in the test subject, and/or multiclass, indicating one or more particular cancer types from a plurality of screened cancer types. In particular, a cancer classifier may be a machine-learned model comprising a plurality of classification parameters and a function representing a relation between the feature vector as input and the cancer prediction as output. Inputting the feature vector into the function with the classification parameters can yield the cancer prediction. In one or more embodiments, an age prediction model is used to predict an age of an individual associated with the test sample based on methylation features. A residual of the predicted age and a reported age of the test subject may be utilized as a feature in the cancer classifier. In one or more embodiments, the feature vectors input into the cancer classifier are based on a set of anomalous fragments (also referred to as “anomalously methylated” or “unusual fragments of extreme methylation” (UFXM)) determined from the test sample. The anomalous fragments may be determined via the process 520 in FIG. 5B, or more specifically hypermethylated and hypomethylated fragments as determined via the step 570 of the process 520, or anomalous fragments determined according to some other process. Prior to deployment of the cancer classifier, the analytics system can train the cancer classifier.

Cancer classification can involve extraction of genetic features and applying one or more models to the extracted features to determine a cancer prediction. The extracted features can include a feature vector for a test sample and determine a cancer prediction based on the input feature vector. The cancer prediction may comprise a label and/or a value. The label may be binary, indicating a presence or absence of cancer in the test subject, and/or multiclass, indicating one or more particular cancer types from a plurality of screened cancer types. In particular, a cancer classifier may be a machine-learned model comprising a plurality of classification parameters and a function representing a relation between the feature vector as input and the cancer prediction as output. Inputting the feature vector into the function with the classification parameters can yield the cancer prediction. In one or more embodiments, an age prediction model is used to predict an age of the test sample based on methylation features. A residual of the predicted age and a reported age of the test subject may be utilized as a feature in the cancer classifier. In one or more embodiments, the feature vectors input into the cancer classifier are based on a set of anomalous fragments (also referred to as “anomalously methylated” or “unusual fragments of extreme methylation” (UFXM)) determined from the test sample. The anomalous fragments may be determined via the process 520 in FIG. 5B, or more specifically hypermethylated and hypomethylated fragments as determined via the step 570 of the process 520, or anomalous fragments determined according to some other process. Prior to deployment of the cancer classifier, the analytics system can train the cancer classifier.

### III.A. Age Prediction Model

The age prediction model can predict an age of a sample based on methylation features extracted from the methylation patterns in the sample. The age prediction model may evaluate methylation features over a plurality of age-informative or age-indicative genomic regions. The genomic regions may be single CpG sites or regions covering multiple CpG sites. Methylation features can be derived from the methylation patterns of the sequence reads in the sample. The number of age-indicative regions, and thereby age-indicative features, may be 1, 5, 10, 25, 50, 100, 1,000, 10,000, 100,000, or more genomic regions.

FIG. 3A illustrates methylation features that can be derived from a single CpG site 305 as a genomic region, according to one or more embodiments. For example, there are six fragments 310 that overlap the single CpG site 305. At each CpG site on a fragment, denoted as a diamond, the fragment has a methylation state. Methylation states may include methylated, shown as filled in, unmethylated shown as unfilled, and variant shown with a diagonal hatch. Variant methylation may include indeterminate states caused from mutations or sequencing errors. One methylation feature is a methylation density at the CpG site 305. In this example, four out of six fragments (fragment 1 310A, fragment 2 310B, fragment 5 310E, and fragment 6 310F) have a methylated status at the CpG site 305. The methylation density would be 4/6, 0.66, or 66%. Another methylation feature counts a percentage of highly methylated fragments that overlap the CpG site 305. Highly methylated fragments may have an above-threshold percentage of methylation at the overlapping CpG sites. Example threshold percentages include 75%, 80%, 85%, 90%, 95%, etc. In this example, using an 80% threshold percentage, three out of six fragments (fragment 1 310A, fragment 2 310B, and fragment 3 310C) are highly methylated, having at least four out of five CpG sites methylated. The percentage of overlapping highly methylated fragments would be 3/6, 0.50, or 50%. Another methylation feature counts a percentage of highly unmethylated fragments overlapping the CpG site 305. Example threshold percentages include 75%, 80%, 85%, 90%, 95%, etc. In this example, taking 80% as the threshold percentage, one out of six fragments (fragment 310B) is highly unmethylated, having at least four out of five CpG sites unmethylated. The percentage of overlapping highly unmethylated fragments would be 1/6, 0.16, or 16%. Fragment 5 310E and fragment 6 310F have mixed methylation, i.e., not highly methylated nor highly unmethylated. Utilizing the highly methylated or unmethylated methylation features aims to identify more important fragments that overlap the CpG site 305. Taking fragment 3 310C as an example, fragment 3 310C is unmethylated at the CpG site 305, but as a highly methylated fragment, fragment 3 310C contributes to the count of highly methylated overlapping fragments. Other methylation features may be derived based on the above noted methylation features. For example, another methylation feature may involve a comparison between the count of highly methylated overlapping fragments and the count of highly unmethylated overlapping fragments, e.g., a ratio between the two counts.

FIG. 3B illustrates methylation features that can be derived from multiple CpG sites as a genomic region 315, according to one or more embodiments. The CpG sites 317 include CpG sites 1, 2, 3, 4, and 5. As in FIG. 3A, a filled diamond indicates methylation, an unfilled diamond indicates unmethylation, and a diagonal hatch diamond indicates variant. Akin to FIG. 3A, methylation features include, but are not limited to, methylation density across the genomic region 315, percentage of highly methylated fragments overlapping the genomic region 315, and percentage of highly unmethylated fragments overlapping the genomic region 315. For methylation density, the methylated states over the CpG sites 317 over all fragments 420 is divided by the total CpG sites on the fragments 320. In this example, the methylation density is 0.63 or 63%. Fragment 1 320A, fragment 2 320B, and fragment 3 320C are highly methylated, above 80% methylation across the fragment (at least as shown). Fragment 4 320D is highly unmethylated, above 80% unmethylation across the fragment (at least as shown). Fragment 5 320E and fragment 6 320F are mixed methylation, i.e., neither highly methylated nor highly unmethylated. As such, one methylation feature as the percentage of highly methylated fragments overlapping the genomic region 315 is 0.50 or 50%. Another methylation feature as the percentage of highly unmethylated fragments overlapping the genomic region 315 is 0.17 or 17%. A fragment overlapping the genomic region 315 may be a fragment that overlaps at least one of the CpG sites 317. In some embodiments, fragments overlapping the genomic region 315 overlap at least some percentage of the CpG sites 317, e.g., at least 20% of the CpG sites 317 of the genomic region 315.

FIG. 4A illustrates training 400 of an age prediction model, according to one or more embodiments. The analytics system may perform some or all of the training 400. In other embodiments, other components in FIGS. 6A & 6B may perform some or all of the training 400. The training 400 yields a trained age prediction model, which may input methylation features for a set of age informative genomic regions and output a predicted age. The process of training 400 the age prediction model can be similarly applied to training other covariate prediction models. In embodiments with other covariates, the analytics system utilizes training samples with reported values for the covariate prediction model being trained.

The analytics system obtains 405 a plurality of training samples. The plurality of training samples can include (1) only cancer training samples, (2) only non-cancer training samples, or (3) a combination of cancer and non-cancer training samples. Each cancer training sample is taken from an individual confirmed to have a cancer diagnosis. The confirmation of the cancer diagnosis can occur before or after the sample is taken. In some examples, the type of cancer may be known. Each non-cancer training sample is taken from an individual free from any cancer diagnosis and may generally be regarded as a healthy individual. Each training sample comprises genetic material that can be sequenced and analyzed. In some embodiments, the samples are blood samples comprising nucleic acid fragments, e.g., cfDNA fragments. Additionally, each training sample includes a chronological age reported by the individual. That is, each training sample is labeled with the chronological age of the subject from whom the sample was obtained. For instance, if a cancer subject is obtained from a 39 year-old male, that cancer training sample will be labelled as being obtained from a 39 year-old male. In some cases, labels may include errors (e.g., due to sample swaps between subjects). For instance, a non-cancer training sample may be labelled as being obtained from a 62 year-old woman when, in fact, it was obtained from a 76 year-old man.

The analytics system sequences 410 the nucleic acid fragments in each training sample to identify a methylation pattern for each nucleic acid fragment. Methylation patterns, again, represent the methylation state of CPG sites in DNA fragments within a genomic region in a sample. Methylation patterns may be determined relative to other samples or a population of samples. Sequencing may involve bisulfite sequencing to convert unmethylated CpG sites. In other embodiments, a sequencer performs the sequencing of the nucleic acid fragments, and the analytics system processes the sequence reads to determine the methylation pattern. The analytic system may further perform one or more processing steps to the sequence reads, e.g., de-duplicating copies of the same original fragment, identifying 38 contaminated fragments, identifying sequencing error, etc. The process of sequencing the nucleic acid fragments and determining a methylation pattern is discussed above in FIGS. 2A & 2B.

For each genomic region, the analytics system calculates 415 an indicativeness score between chronological age and the methylation patterns of nucleic acid fragments overlapping the genomic region. More generally, the indicativeness score represents the correlation between the chronological age of subjects and methylation patterns. The analytics system may determine one or more methylation features for each genomic region of an initial set of genomic regions. The initial set of genomic regions may be an expansive set covering a majority of CpG sites in the human genome. For example, the analytics system for a single CpG site genomic region may determine some combination of methylation features described in FIG. 3A, e.g., some combination of methylation density, percentage of overlapping highly methylated fragments, and percentage of overlapping highly unmethylated fragments. The analytics system can calculate an indicativeness score for a genomic region by training a regression of chronological age based on the methylation features at that genomic region. The analytics system trains the regression using methylation features extracted for each training sample and the reported age of the training sample (e.g., the chronological age with which the sample is labelled). Types of regressions include linear regression, logarithmic regression, exponential regression, multivariate regression, logistic regression, polynomial regression, lasso regression, etc. From the trained regression, the analytics system may measure various metrics for use as an indicativeness score. Example metrics may include, but are not limited to, a covariance, Pearson's correlation coefficient (or simply “Pearson's correlation”), R2, sum of squares of residuals (RSS), total sum of squares (TSS), a t-statistic of the slope, two-tailed p-value of the t-statistic (which may be adjusted, e.g., for multiple hypothesis testing), other statistical metrics relating to regressions, etc.

To illustrate, the analytics system at a genomic region calculates a multivariate chronological age regression based on the methylation density, the percentage of overlapping highly methylated fragments, and the percentage of overlapping highly unmethylated fragments, and the like. Based on the trained multivariate regression, the analytics system may determine a Pearson's correlation that may serve as the indicativeness score for the genomic region. In another example, the analytics system at a genomic region calculates a linear chronological age regression based solely on the methylation density, and may determine the Pearson's correlation as the indicativeness score. In some embodiments, the analytics system may train multiple regressions for each genomic region based on varying sets of methylation features considered, e.g., a first regression based solely on the methylation density, a second regression based solely on the percentage of overlapping highly methylated fragments, a third regression based solely on the percentage of overlapping highly unmethylated fragments, a fourth regression based on a combination of multiple of the above three methylation features.

The analytics system generates 420 a feature set of genomic regions based on the covariance scores for use as features in the age prediction model. To do so, the analytics system may calculate one or more indicativeness scores for each of the initial set of genomic regions, e.g., one indicativeness score for each set of methylation features. The set of methylation features that achieves the highest absolute indicativeness score at a genomic region is determined to be the most-informative set of methylation features for that genomic region. In one embodiment, the analytics system uses a threshold absolute indicativeness score to identify the genomic regions to use as part of the feature set of genomic regions. For example, the threshold absolute indicativeness score is 0.5, thus indicativeness scores above 0.5 or below −0.5 (e.g., absolute of the indicativeness score is above 0.5) surpass the threshold absolute indicativeness score. In turn, the analytics system generates feature vectors including the identified genomic regions having indicativeness scores above the threshold. For example, the feature vector may include all genomic regions whose absolute indicativeness scores are above 0.5 or below −0.5. In another embodiment, the analytics system ranks the genomic regions based on their highest indicativeness score. From the ranking, the analytics system may select sufficient genomic regions that exhausts a budget of genomic regions, e.g., that can be targeted by an assay panel and generates feature vectors accordingly.

In additional embodiments, the analytics system further considers one or more other factors of the genomic regions to determine the feature set of genomic regions, which are thereby included in generated feature vectors. For example, the analytics system may further consider a balance of negatively correlated and positively correlated genomic regions. The negatively correlated genomic regions reflect increase in value of the methylation features correlated to a decrease in value of age. The positively correlated genomic regions reflect an increase in value of the methylation features correlated to an increase in value of age. The analytics system may further consider a rate of change between age and the methylation features. For example, some genomic regions may be flatly correlative of age (small differentials in methylation features over the age of individuals), some genomic regions may be steadily correlative of age (medium differentials in methylation features over the age of individuals), and some genomic regions may be sharply correlative of age (large differentials in methylation features over the age of individuals). The analytics system may further consider the position of the genomic regions in the human genome, e.g., ensuring a proper spread of the genomic regions across the human genome. This prevents situations with all the age-informative genomic regions being localized to one section of the human genome, which for any number of reasons may have sparse signal in a testing sample for which the age prediction is to be applied, which would frustrate the age prediction if all the age-informative genomic regions were in that section.

In some embodiments, the analytics system identifies a features set of genomic regions utilizing a penalized regression and generates the feature vectors accordingly. The penalization process aims to optimize the set of features utilized to a minimum set of features that still provides optimal predictive power. Other embodiments achieve a similar result utilizing a relaxed lasso regression.

In some embodiments, the analytics system reduces 425 the feature set of genomic regions to genomic regions that have high correlation to cancer signal. That is, the analytics system may remove, or not include, genomic regions from a feature vector or feature set that are not highly correlated to cancer signal. For instance, the analytics system may separately identify genomic regions correlated with cancer signal (or another disease signal). The analytics system may then determine the genomic regions that intersect correlation to age and correlation to cancer signal. One method of identifying genomic regions correlative with cancer signal is disclosed below (e.g., in conjunction with FIGS. 6A & 6B). Utilizing genomic regions that intersect correlation to age and correlation to cancer signal can more efficiently utilize budget for targeted regions on an assay panel. That is, creating a panel with fewer probes that target regions that are more indicative of age and/or cancer presence. In essence, a probe in a panel that targets regions that are strongly correlated to cancer and/or age is more “valuable” to include in a panel than a probe that is weakly correlated to cancer and/or age. Moreover, utilizing intersecting genomic regions may prove advantageous in utilizing predicted age as a feature in cancer classification.

At this juncture, the analytics system has identified a feature set of genomic regions for use in training the age prediction model and generated the corresponding feature vectors. Each genomic region in the feature set (included in a feature vector) may comprise a single CpG site, may comprise multiple CpG sites, or may comprise multiple sets of CpG sites. Each genomic region in the feature set has a particular set of methylation features, which may be different from other genomic regions. For example, a first genomic region covers a single CpG site and considers methylation density at that CpG site, and a second genomic region covers multiple CpG sites and considers both a percentage of overlapping highly methylated fragments and a percentage of overlapping highly unmethylated fragments. In other embodiments, the analytics system implements a penalization to minimize the number of genomic regions that maintain age prediction accuracy. The penalization is a factor that negatively affects the age prediction based on the number of genomic regions. The penalization forces the analytics system to determine a minimum number of genomic regions that maintains optimal performance of the age prediction model.

The analytics system trains 430 the age prediction model based on the methylation patterns of the nucleic acid fragments from the training samples that overlap the feature set of genomic regions. For each training sample, the analytics system determines values for the methylation features of the feature set of genomic regions. In some embodiments, the analytics system trains the age prediction model as a machine-learned model. Example machine-learned models include linear regression, logarithmic regression, exponential regression, multivariate regression, logistic regression, polynomial regression, lasso regression, etc. The analytics system may train multiple age prediction models with varying feature sets of genomic regions to evaluate performance across the various models. For example, a first model is trained on a small feature set of genomic regions, and a second model is trained on a large feature set of genomic regions inclusive of the small feature set. The analytics system evaluates performance of the two age prediction models using a validation set of training samples.

FIG. 4B illustrates deployment 440 of a chronological age prediction model, according to one or more embodiments. The analytics system may perform some or all of the deployment 440. In other embodiments, other components in FIGS. 6A & 6B may perform some or all of the deployment 440. Deployment 440 of the age prediction model includes determining an age prediction of an individual associated with a test sample based on methylation features for the feature set of genomic regions for the test sample.

The analytics system obtains 445 a test sample with a plurality of nucleic acid fragments and a reported age (e.g., a label of the chronological age of the subject from whom the sample is obtained). A physician or other medical provider collects the test sample and may also obtain the reported age of the individual providing the test sample. In some embodiments, age may be a single value or may be an age range. For example, the individual may report an age of 47, or may report an age range of 40-50. The sample may be any type of biological sample comprising nucleic acid material of the individual. In embodiments with a blood sample, the blood sample comprises at least cfDNA fragments sheared from cells.

The analytics system sequences 450 the nucleic acid fragments in the test sample to identify a methylation pattern for each nucleic acid fragment. Sequencing may involve bisulfite sequencing to convert unmethylated CpG sites. In other embodiments, a sequencer performs the sequencing of the nucleic acid fragments, and the analytics system processes the sequence reads to determine the methylation pattern. The analytic system may further perform one or more processing steps to the sequence reads, e.g., de-duplicating copies of the same original fragment, identifying contamination fragments, identifying sequencing errors, etc. The process of sequencing the nucleic acid fragments and determining a methylation pattern is discussed above in FIGS. 2A & 2B.

The analytics system applies 455 the trained age prediction model to predict a chronological age for the test sample based on the methylation patterns of the nucleic acid fragments of the test sample. The analytics system determines methylation features for the age prediction model, e.g., trained by the process 400 of FIG. 4A. The age prediction model is configured to receive as input methylation features for a feature set of genomic regions and output a predicted chronological age based on the methylation features. As with reported age, the predicted age may be a single value or an age range.

The analytics system compares 460 the predicted age to the reported age. The comparison may be a determination of whether the predicted age matches to the reported age. For example, if the reported age was an age range of 20-30, and the predicted age was 26 or the range 20-30, then the predicted age matches to the reported age range. In some embodiments, the comparison may be a residual as a difference between the predicted age and the reported age. For example, if the reported age was 63 and the predicted age was 72, then the residual is 9 years over the reported age. The residual may also be absolute, e.g., 9 years different than the reported age.

The analytics system proceeds with analyses using the predicted age. In some embodiments, the analytics system may perform 465 sample swap validation. Sample swap validation, in this context, can refer to identifying whether the test sample was correctly labelled. For instance, a “sample-swap” may occur if a sample obtained from a 54 year-old woman is labelled as a sample obtained from a 24 year old man. More generally, however, identifying sample swaps is akin to identifying that the reported age of the test sample is incorrect.

In some configurations, the analytics system may utilize the residual to determine whether the sample was swapped in order to determine that the test sample doesn't truly originate from the individual expected to be associated with the sample. The analytics system may call or identify a sample swap if the predicted age is different than the reported age. In other embodiments, the analytics system may call the sample swap if the residual is above a threshold difference. For example, the residual threshold can be set at 10 years, so if the residual between the predicted age and the reported age is above the 10-year residual threshold, then the analytics system can call the sample swap. In yet other embodiments, the analytics system may call the sample swap based on the comparison of the predicted age and the reported age in conjunction with other analyses. For example, the analytics system may train a separate model for ethnicity determination to determine whether a predicted ethnicity matches to the individual's reported ethnicity or a separate model for sex determination to determine whether a predicted sex matches to the individual's reported sex. Upon calling a sample swap, the analytics system may withhold the sample from downstream analyses. Samples not called to be sample swaps may proceed with downstream analyses. For example, upon calling a sample swap for a training sample, the analytics system may withhold the training sample from use in training one or more models or building one or more distributions. As another example, upon calling a sample swap for a test sample, the analytics system may withhold cancer prediction for the test sample.

The analytics system may use 470 the comparison of the predicted age to the reported age as part of cancer classification. In one or more embodiments, the analytics system uses the residual of the predicted age to the reported age as a feature to cancer classification, e.g., in conjunction with other features extracted from the sequencing data. For instance, a residual may indicate that there is an abnormally high or low amount of methylation in a test sample (leading to a high residual), which, as described above, is indicative of cancer presence or non-presence. As such, a high residual may thereby be used as an indicative feature in determining whether a test sample has cancer.

In some embodiments, the analytics system may compare the residual to a residual threshold for determining whether the sample has a strong likelihood for presence of cancer. The analytics system may set the residual threshold using a set of training samples. The analytics system identifies methylation features for the feature set of genomic regions for each training sample. The analytics system inputs the methylation features for each training sample into the age prediction model to determine a predicted age for each training sample. The analytics system may calculate a residual for each training sample by calculating a difference between the predicted age and the reported age. The analytics system may set the residual threshold that encompasses a significant majority of the training samples. For example, the analytics system wants to utilize a residual threshold that captures 75%, 80%, 85%, 90%, 95%, 97%, 98%, 99%, 99.5%, or 99.9% of the training samples. In effect, setting the residual threshold is training the model to recognize samples with methylation patterns that are causing an error in chronological age prediction to be associated with cancer presence or non-cancer presence. Thus, the residual threshold is used as a measure to determine whether an amount of difference seen in a methylation pattern is attributable to age, or if it is attributable to cancer. Given this, if a test sample has a residual that is outside of the residual threshold, the analytics system can make an initial determination that the test sample has a strong likelihood for presence of cancer. Depending on the configuration, strong likelihood can indicate that the sample is more likely than not to indicate cancer, have a probability of including cancer of, e.g., at least 60%, 65%, 70%, 80%, 90%, etc., or may have an indicativeness score above a threshold. The analytics system can proceed with cancer classification to corroborate the initial determination.

### III.B. Identifying Anomalous Fragments

The analytics system can determine anomalous fragments for a sample using the sample's methylation state vectors. For each fragment in a sample, the analytics system can determine whether the fragment is an anomalous fragment using the methylation state vector corresponding to the fragment. In some embodiments, the analytics system calculates a p-value score for each methylation state vector describing a probability of observing that methylation state vector or other methylation state vectors even less probable in the healthy control group. In some examples, the p-value score may be adjusted, e.g., for multiple hypothesis testing by controlling for a false positive rate, a family-wise error rate, a false discovery rate, etc. The process for calculating a p-value score is further discussed below in Section II.C.i. P-Value Filtering. The analytics system may determine fragments with a methylation state vector having below a threshold p-value score as anomalous fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### II.C.I. P-Value Filtering

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be anomalously methylated, the analytics system can use a healthy control group with a majority of fragments that are normally methylated. When conducting this probabilistic analysis for determining anomalous fragments, the determination can hold weight in comparison with the group of control subjects that make up the healthy control group. To ensure robustness in the healthy control group, the analytics system may select some threshold number of healthy individuals to source samples including DNA fragments. FIG. 5A below describes the method of generating a data structure for a healthy control group with which the analytics system may calculate p-value scores. FIG. 5B describes the method of calculating a p-value score with the generated data structure.

FIG. 5A is a flowchart describing a process 500 of generating a data structure for a healthy control group, according to an embodiment. To create a healthy control group data structure, the analytics system can receive a plurality of DNA fragments (e.g., cfDNA) from a plurality of healthy individuals. The analytics system can generate 505 a methylation state vector for each fragment, for example via the process 200.

With each fragment's methylation state vector, the analytics system can subdivide 510 the methylation state vector into strings of CpG sites. In some embodiments, the analytics system subdivides 510 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 can result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 515 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 510 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 515 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is anomalous or not.

FIG. 5B is a flowchart describing a process 530 for identifying anomalously methylated fragments from an individual, according to an embodiment. In process 530, the analytics system generates 540 methylation state vectors from cfDNA fragments of the subject, e.g., via the process 200. The analytics system can handle each methylation state vector as follows.

For a given methylation state vector, the analytics system enumerates 545 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2ⁿ possibilities of methylation state vectors. With methylation state vectors inclusive of indeterminate states for one or more CpG sites, the analytics system may enumerate 530 possibilities of methylation state vectors considering only CpG sites that have observed states.

The analytics system calculates 550 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMMs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 555 a p-value score for the methylation state vector using the calculated probabilities for each possibility. In some embodiments, this includes identifying the calculated probability corresponding to the possibility that matches the methylation state vector in question. Specifically, this can be the possibility of having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy individual, and which causes the fragment to be labeled anomalously methylated, relative to the healthy control group. A high p-value score can generally relate to a methylation state vector that is expected to be present, in a relative sense, in a healthy individual. If the healthy control group is a non-cancerous group, for example, a low p-value can indicate that the fragment is anomalously methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are anomalously methylated, the analytics system may filter 565 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 500, the analytics system can yield a median (range) of 2,800 (1,500-12,000) fragments with anomalous methylation patterns for participants without cancer in training, and a median (range) of 3,000 (1,200-420,000) fragments with anomalous methylation patterns for participants with cancer in training. These filtered sets of fragments with anomalous methylation patterns may be used for the downstream analyses as described below in Section III.

In some embodiments, the analytics system uses 560 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytic system can calculate a p-value score for the window including the first CpG site. The analytics system can then “slide” the window to the second CpG site in the vector, and calculates another p-value score for the second window. Thus, for a window size/and methylation vector length m, each methylation state vector can generate m−l+1 p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10 \hat{ }3) probability calculations. This can result in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

In some examples, the p-value scores may be adjusted for multiple hypothesis testing according to a variety of suitable techniques. As an example, and without limitation, said techniques can include controlling for false positive rate, family-wise error rate, experiment-wise error rate, false discovery rate, etc. Known and applicable techniques for adjusting p-values include, without limitation, a Bonferroni procedure, a Holm procedure, a Hochberg procedure, a harmonic mean p-value procedure, a Benjamini-Hochberg procedure, a Benjamini-Yekutieli procedure, a Storey-Tibshirani procedure, etc. Adjusting p-value scores for multiple hypothesis testing can be used to improve the accuracy associated with basing positive detection calls based on the p-value and to reduce the incidence of false positives.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of 1's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. Provisional patent application Ser. No. 17/191,914, titled “Systems and Methods for Cancer Condition Determination Using Autoencoders,” filed Mar. 4, 2021, which is hereby incorporated herein by reference in its entirety.

The respective nucleic acid methylation fragment may fail to satisfy a selection criterion in the one or more selection criteria when the respective nucleic acid methylation fragment has an anomalous methylation score that is less than an anomalous methylation score threshold. In this situation, the anomalous methylation score can be determined by a mixture model. For example, a mixture model can detect an anomalous methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An anomalous methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 4, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 4, 5, or more than 5. For example, a first nucleic acid methylation fragment having the same corresponding genomic start and end position as a second nucleic acid methylation fragment but having at least 1, at least 2, at least 3, at least 4, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

### III.C.II. Hypermethylated Fragments and Hypomethylated Fragments

In some embodiments, the analytics system identifies 570 determines hypomethylated fragments or hypermethylated fragments from the filtered set as anomalous fragments. The analytics system identifies hypermethylated fragments having over a threshold number of CpG sites and over a threshold percentage of the CpG sites methylated. The analytics system identifies hypomethylated fragments having over the threshold number of CpG sites and over a threshold percentage of CpG sites unmethylated. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### III.C. Training of Cancer Classifier

FIG. 6A is a flowchart describing a process 600 of training a cancer classifier, according to an embodiment. The analytics system obtains 610 a plurality of training samples each having a set of anomalous fragments and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 620, for each training sample, a feature vector based on the set of anomalous fragments of the training sample. The analytics system can calculate an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5. In one or more embodiments, the feature vector further includes one or more features based on a chronological age prediction (e.g., covariate prediction) described in FIGS. 4A & 4B. For example, the feature vector may include an age residual as a difference between a predicted chronological age (e.g., via the process 440 by applying a trained age prediction model) and a reported chronological age. In other examples, the feature vector may include other features based on the predicted covariates, e.g., one or more of the predicted covariates. In some embodiments, the feature vector further includes one or more methylation features from the feature set evaluated in the chronological age prediction (e.g., the feature set of the chronological age prediction model determined at step 420 in FIG. 4A).

Once all anomaly scores are determined for a training sample, the analytics system can determine the feature vector as a vector of elements including, for each element, one of the anomaly scores associated with one of the CpG sites in an initial set. The analytics system can normalize the anomaly scores of the feature vector based on a coverage of the sample. Here, coverage can refer to a median or average sequencing depth over all CpG sites covered by the initial set of CpG sites used in the classifier, or based on the set of anomalous fragments for a given training sample.

As an example, reference is now made to FIG. 6B illustrating a matrix of training feature vectors 622. In this example, the analytics system has identified CpG sites [K] 626 for consideration in generating feature vectors for the cancer classifier. The analytics system selects training samples [N] 624. The analytics system determines a first anomaly score 628 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 628 for the first CpG site as 1, as illustrated in FIG. 6B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 629 for the second CpG site [k2] to be 0, as illustrated in FIG. 6B. Once the analytics system determines all the anomaly scores for the initial set of CpG sites, the analytics system determines the feature vector for the first training sample [n1] including the anomaly scores with the feature vector including the first anomaly score 628 of 1 for the first CpG site [k1] and the second anomaly score 629 of 0 for the second CpG site [k2] and subsequent anomaly scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety.

The analytics system may further limit the CpG sites considered for use in the cancer classifier. The analytics system computes 630, for each CpG site in the initial set of CpG sites, an information gain based on the feature vectors of the training samples. From step 620, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 630 an information gain for each cancer type and for each CpG site in the initial set to determine whether to include that CpG site in the classifier. The information gain is computed for training samples with a given cancer type compared to all other samples. For example, two random variables ‘anomalous fragment’ (‘AF’) and ‘cancer type’ (‘CT’) are used. In one embodiment, AF is a binary variable indicating whether there is an anomalous fragment overlapping a given CpG site in a given samples as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. In practice, for a first cancer type, the analytics system computes pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly anomalously methylated in training samples of a given cancer but not in training samples of other cancer types or in healthy training samples, then CpG sites overlapped by those anomalous fragments can have high information gains for the given cancer type. The ranked CpG sites for each cancer type can be greedily added (selected) 640 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 650 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 620 or to the selected set of CpG sites from step 650. In one embodiment, the analytics system trains 660 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 670 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

### III.D. Deployment of Cancer Classifier

During use of the cancer classifier, the analytics system can obtain a test sample from a subject of unknown cancer type. The analytics system may process the test sample comprised of DNA molecules with any combination of the processes 200 and 530 to achieve a set of anomalous fragments. The analytics system can determine a test feature vector for use by the cancer classifier according to similar principles discussed in the process 600. The analytics system can calculate an anomaly score for each CpG site in a plurality of CpG sites in use by the cancer classifier. For example, the cancer classifier receives as input feature vectors inclusive of anomaly scores for 1,000 selected CpG sites. The analytics system can thus determine a test feature vector inclusive of anomaly scores for the 1,000 selected CpG sites based on the set of anomalous fragments. The analytics system can calculate the anomaly scores in a same manner as the training samples. In some embodiments, the analytics system defines the anomaly score as a binary score based on whether there is a hypermethylated or hypomethylated fragment in the set of anomalous fragments that encompasses the CpG site. In some embodiments, the analytics system performs covariate prediction (e.g., the process 440 in FIG. 4B) to predict a covariate value and/or label. The analytics system may generate the test feature vector including one or more features based on the covariate prediction.

The analytics system can then input the test feature vector into the cancer classifier. The function of the cancer classifier can then generate a cancer prediction based on the classification parameters trained in the process 600 and the test feature vector. In the first manner, the cancer prediction can be binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 660 of the process 600 with another cancer classifier trained in step 670 or the process 600. The analytics system can input the test feature vector into the cancer classifier trained as a binary classifier in step 660 of the process 600. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

### IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V. Example Results

### V.A. Sample Collection and Processing

Study design and samples: CCGA (NCT02889978) is a prospective, multi-center, case-control, observational study with longitudinal follow-up. De-identified biospecimens were collected from approximately 15,000 participants from 342 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, MD). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, MI) and constructed libraries were quantified using KAPA Library Quantification Kit for Illumina Platforms (Kapa Biosystems; Wilmington, MA). Four libraries along with 10% PhiX v3 library (Illumina, FC-110-3001) were pooled and clustered on an Illumina NovaSeq 7000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.C. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypomethylated). This procedure resulted in a median (range) of 2,800 (1,500-12,000) UFXM fragments for participants without cancer in training, and a median (range) of 3,000 (1,200-420,000) UFXM fragments for participants with cancer in training. As this data reduction procedure only used reference set data, this stage was only required to be applied to each sample once.

### V.B. Co Variate Prediction Results

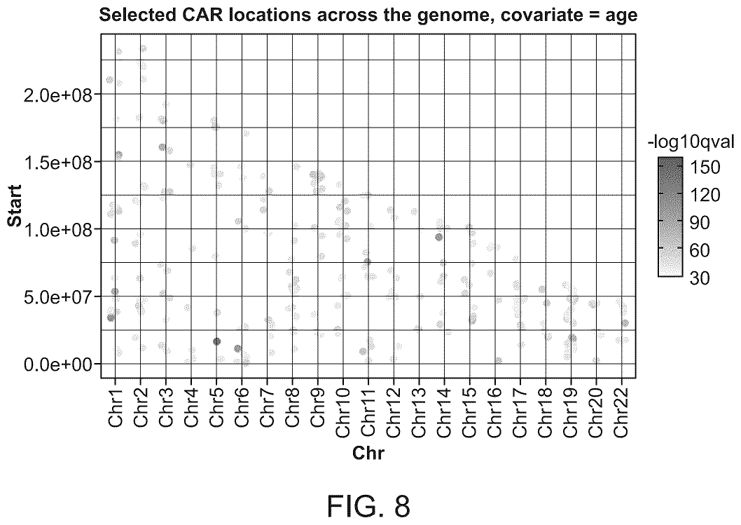

FIG. 8 illustrates genomic regions associated with age, in accordance with one or more example implementations. The analytics system can train regressions using the methylation features determined for training samples. The examples, for illustration purposes, use only non-cancer training samples. In this example, the analytics system calculates a two-tailed p-value for the regression slope from a t-statistic for each genomic region based on the trained linear regressions. A lower p-value indicates a higher unlikeliness of observing that slope, which translates to a more discriminatory genomic region for indicating the chronological age of the subject from whom the sample was obtained. In the graph shown, the x-axis plots the chromosomes in the human body, while the y-axis plots position within each chromosome. Each mark represents a region (CAR) comprising a cluster of genomic regions within 500 CpG sites that have an indicativeness score above some threshold indicativeness score. Each CAR indicates the lowest p-value of genomic regions clustered in the CAR. The legend to the right of the graph is a negative logarithm of the p-value, such that the higher the negative logarithm, the lower the p-value.

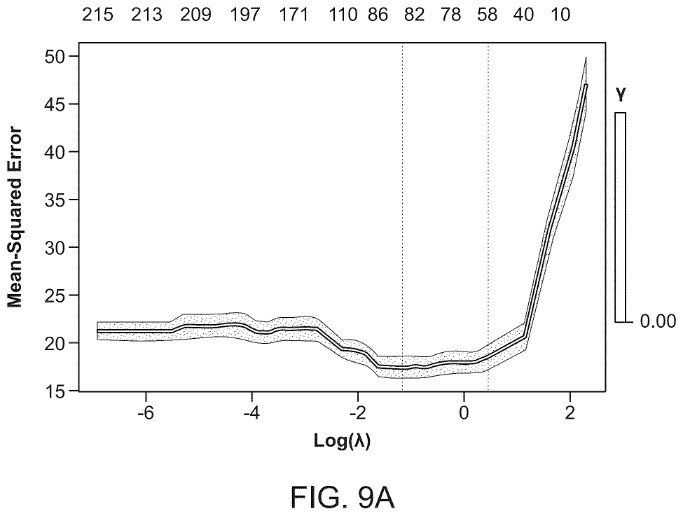

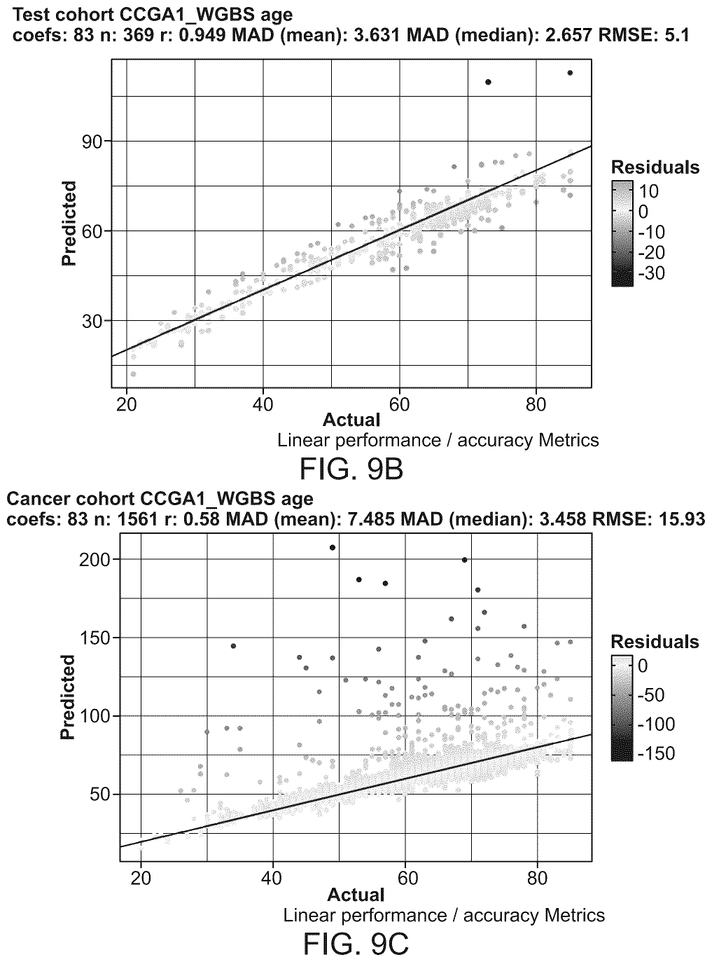

FIG. 9A illustrates one process of identifying a feature set of genomic regions informative of age for use in a generated feature vector, in accordance with one or more example implementations. The training samples utilized were from the CCGA study. The analytics system performs a glmnet relaxed lasso regression on a lambda grid to identify an optimal range of genomic regions to use as the feature set. The optimal set of genomic regions in the range of 58 to 83 genomic regions provided the lowest mean-squared error (plotted on the y-axis). A feature set of 83 genomic regions were used to train the age prediction model for the test sets.

FIGS. 9B and 9C illustrate age residuals using the age prediction model trained on the feature set identified in FIG. 9A, in accordance with example implementations. The x-axis plots reported chronological age (“actual”) against y-axis of predicted chronological age (“predicted”) by the age prediction model. FIG. 9B illustrates a graph of results of age prediction on a non-cancer holdout cohort, in accordance with example implementations. The holdout cohort was not used in the training of the regression. The hold-out cohort had a total of 369 samples. The residual is calculated as a subtracting the predicted age from the reported age. Of note, the highest residuals in the non-cancer cohort were around approximately −35 and +25, with the vast majority of the non-cancer cohort having a residual within −10 and +10. FIG. 9C illustrates a graph of results of age prediction on a cancer cohort, in accordance with example implementations. As the age prediction model is fitted on non-cancer samples, none of the cancer samples have been used in the training of the regression. The cancer cohort had a total of 1561 samples. Here, the highest residual is approximately −155. The spread of the residuals is also way more dispersed compared to the non-cancer cohort in FIG. 9B.

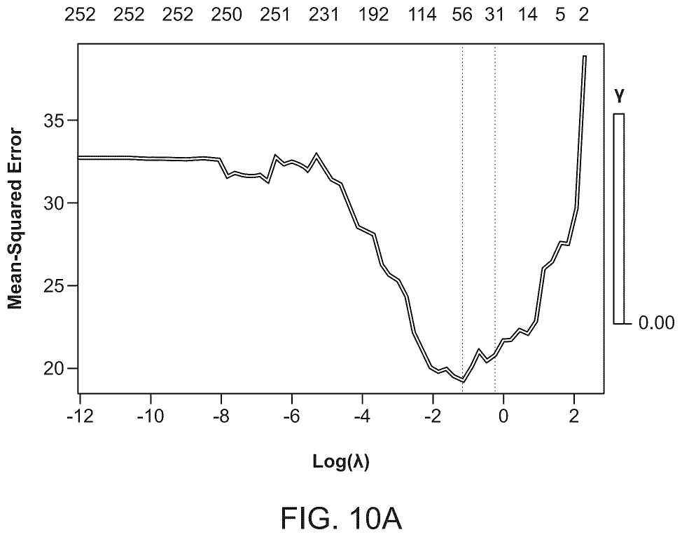

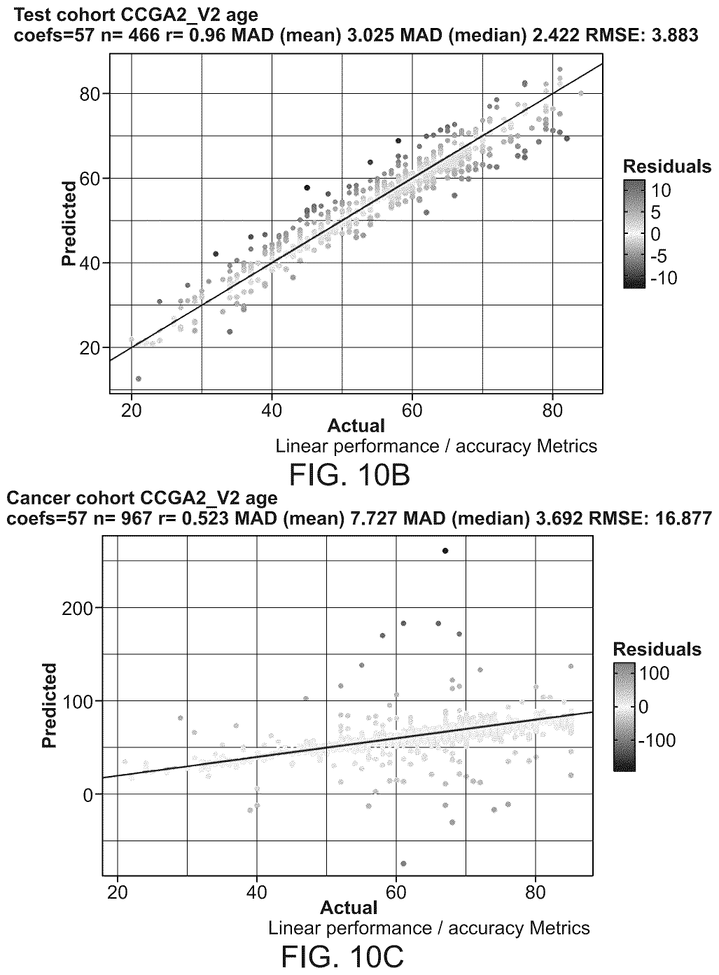

FIG. 10A illustrates another process of identifying a feature set of genomic regions informative of chronological age, in accordance with one or more example implementations. The training samples utilized were from a follow-up to the CCGA study, termed CCGA2. Akin to FIG. 9A, the analytics system performs a glmnet relaxed lasso regression on a lambda grid to identify an optimal range of genomic regions to use as the feature set. The optimal set of genomic regions in the range of 31 to 57 provided the lowest mean-squared error (plotted on the y-axis). A feature set of 57 genomic regions were used to train the age prediction model for the test sets.

FIGS. 10B and 10C illustrate age residuals using the age prediction model trained on the feature set identified in FIG. 10A, in accordance with example implementations. The x-axis plots reported age (“actual”) against y-axis of predicted age (“predicted”) by the age prediction model. FIG. 10B illustrates a graph of results of age prediction on a non-cancer holdout cohort, in accordance with example implementations. The holdout cohort was not used in the training of the regression. The hold-out cohort had a total of 466 samples. The residual is calculated as a subtracting the predicted age from the reported age. Of note, the highest residuals in the non-cancer cohort were around −10 and +10, with the vast majority of the non-cancer cohort having a residual within −5 and +5. FIG. 10C illustrates a graph of results of age prediction on a cancer cohort, in accordance with example implementations. As the age prediction model is fitted on non-cancer samples, none of the cancer samples have been used in the training of the regression. The cancer cohort had a total of 967 samples. Here, the highest residuals are −193 and +135 (on either side of under or over prediction). The spread of the residuals is also more dispersed compared to the non-cancer cohort in FIG. 10B.

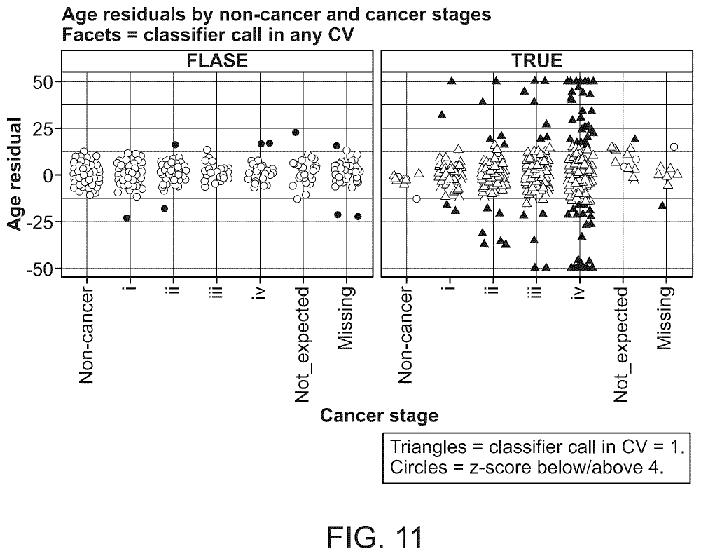

FIG. 11 illustrates the spread of the test cohorts over stages of cancer, in accordance with example implementations. The x-axis of the graphs split out the known cancer state of the samples: non-cancer, stages 1-4 of cancer, with miscellany states (“not expected” and “missing”). The left graph encompasses samples the classifier predicted to not have cancer, i.e., negative results inclusive of both true negatives and false negatives. The right graph encompasses samples the classifier predicted to have cancer, i.e., positive results inclusive of both true positives and false positives. A residual threshold (shown as “z-score” above/below 4) was four standard deviations from the mean. Any sample with chronological age residual above the threshold was colored red with the remainder colored yellow. Two important things to note in the left graph. First, none of the true non-cancer samples had chronological age residuals above the chronological age residual threshold (no red-marked samples). Second, there are a number of cancer samples that were false negatives, but some of those false negatives had chronological age residuals beyond the residual threshold. Utilizing the chronological age residual threshold could have identified these false negatives as having cancer. On the right graph, there are also two important observations. First, a significant number of the cancer samples were above the chronological age residual threshold compared to the non-cancer samples in either graph. Second, later stages of cancer had increasing number of samples with chronological age residuals above the chronological age residual threshold. This indicates an increasing degradation in the chronological age prediction from the sample methylation signature as cancer status accelerates.

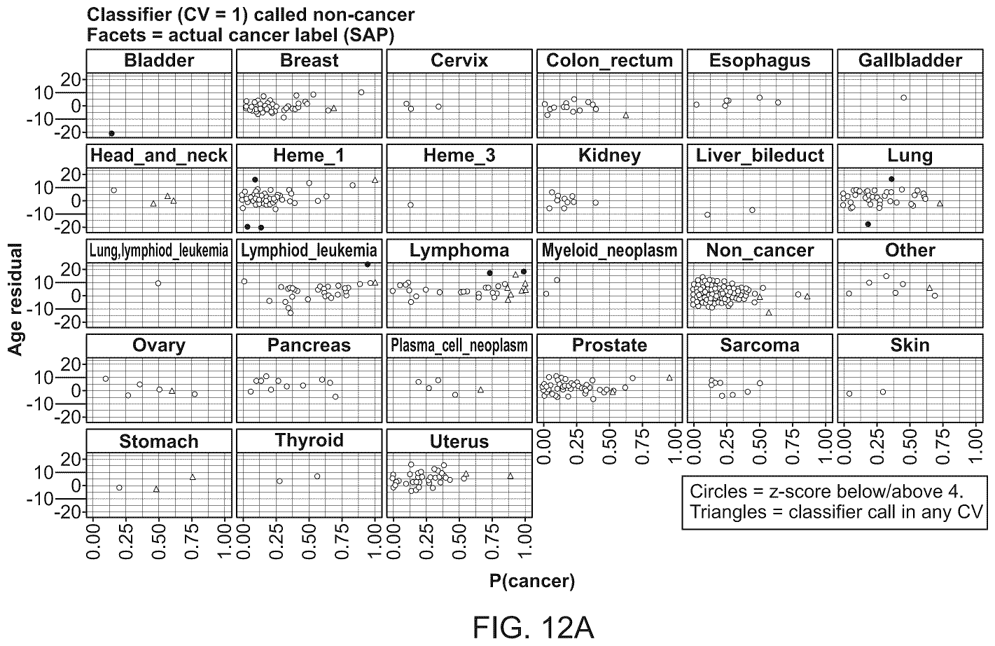

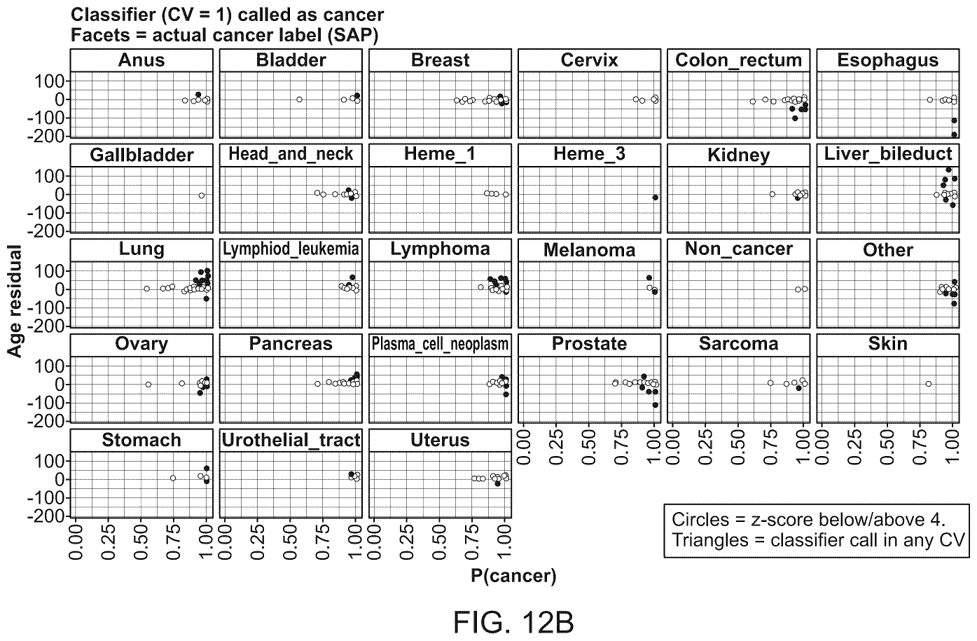

FIGS. 12A and 12B illustrate the spread of the test cohorts over cancer types, in accordance with example implementations. FIG. 12A shows a top series of graphs representing test samples predicted by the cancer classifier to be negative results, i.e., predicted to not have cancer. FIG. 12B shows a bottom series of graphs representing test samples predicted by the cancer classifier to be positive results, i.e., predicted to have cancer. A similar chronological age residual threshold is utilized, e.g., z-score above or below 4. In the top series of graphs, there are at least nine false negative samples (samples known to have cancer but predicted by the cancer classifier to not have cancer) to have chronological age residuals (as calculated by the chronological age prediction model) above the chronological age residual threshold. These are samples that could have been determined to have a strong likelihood of cancer.

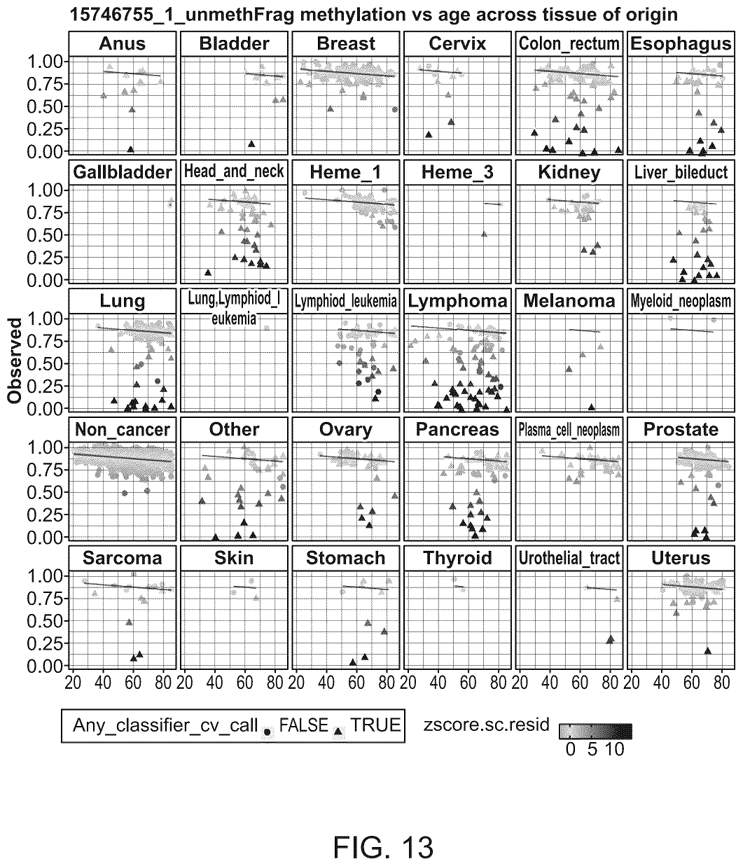

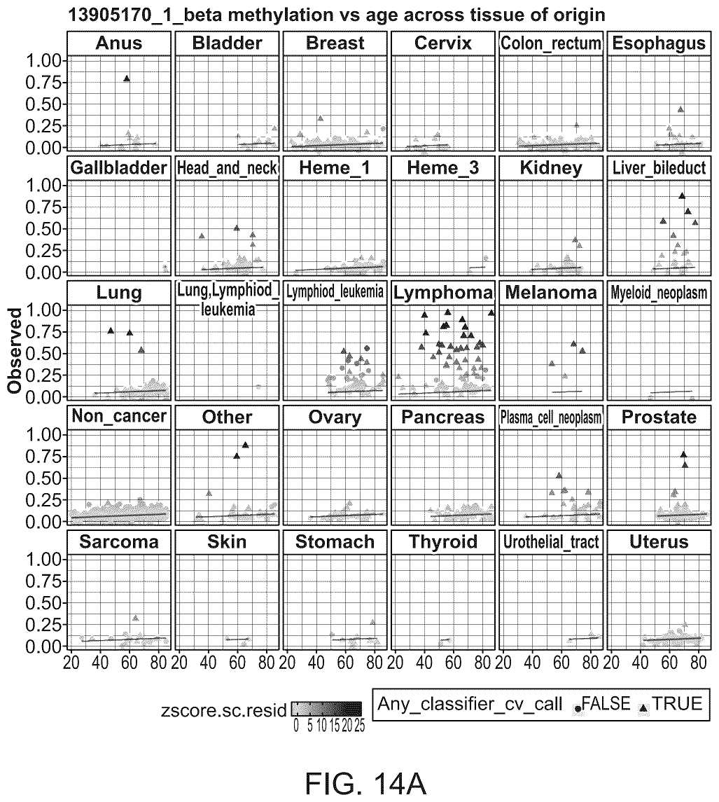

FIG. 13 illustrates one genomic region showing chronological age deceleration of chronological age across cancer types, in accordance with example implementations. The samples are shown are all cancer samples of varying cancer types. Each graph's x-axis shows the true chronological age of the sample with the y-axis showing the predicted chronological age as a fraction of the true age. In numerous cancer types, the predicted chronological age drops off. This genomic region is generally indiscriminate between the cancer types, but decelerates chronological age in most all of the cancer types (with some potentially hindered by low sampling).

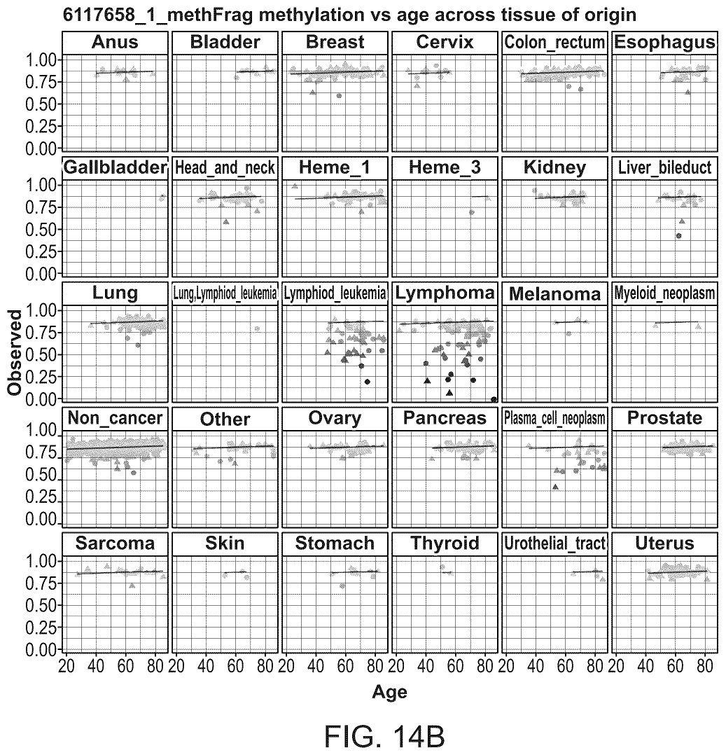

FIGS. 14A and 14B illustrate two genomic regions that are discriminant between hematological cancer types and non-hematological cancer types, in accordance with example implementations. The samples are shown are all cancer samples of varying cancer types. Each graph's x-axis shows the true chronological age of the sample with the y-axis showing the predicted chronological age as a fraction of the true age. FIG. 14A shows results for a first genomic region that appears to be consistently chronological age accelerating in hematological cancer types, with less consistent chronological age acceleration in other cancer non-hematological cancer types. FIG. 14B shows results for a second genomic region that appears to be consistently chronological age decelerating in hematological cancer types, with little to no significant chronological age deceleration in other cancer non-hematological cancer types.

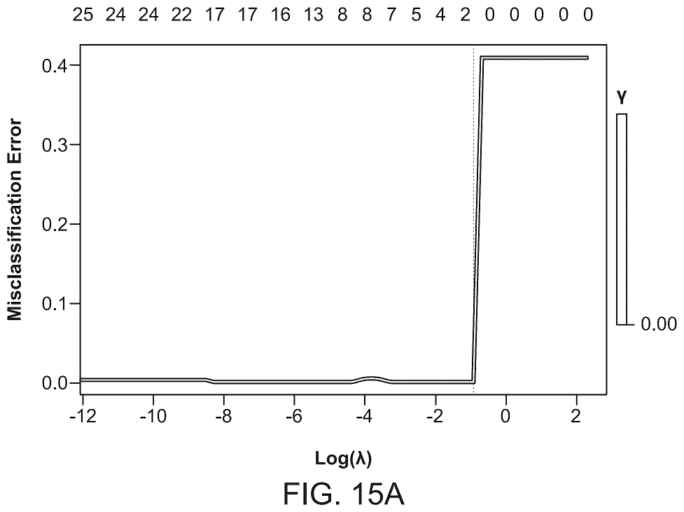

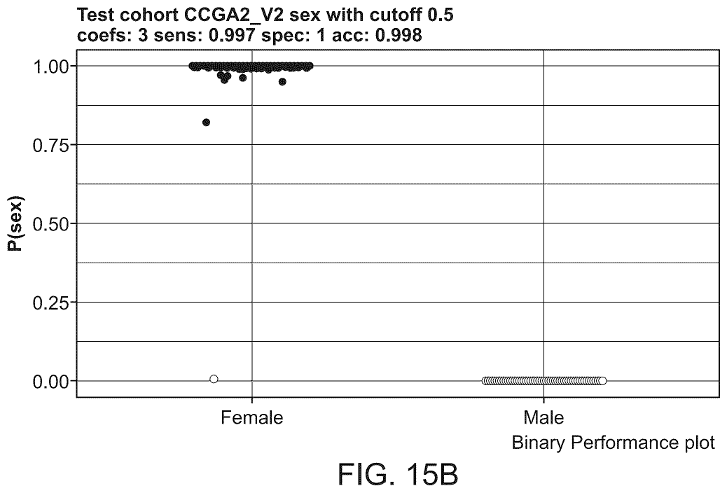

FIG. 15A illustrates identification of a feature set of genomic regions for predicting biological sex, in accordance with one or more example implementations. The training samples utilized were the CCGA2 study. The analytics system performs a glmnet relaxed lasso regression on a lambda grid to identify an optimal range of genomic regions to use as the feature set. The optimal set of genomic regions in the range of 2 or more provided the lowest mean-squared error (plotted on the y-axis). A feature set of 3 genomic regions were used to train the biological sex prediction model for the test sets.

FIG. 15B illustrates results from a trained biological sex prediction model, in accordance with example implementations. The biological sex prediction model was trained on the 3 genomic regions identified in FIG. 15A. The test cohort comprise non-cancer samples. In sum, the biological sex prediction model was 99.8% accurate with 100% specificity.

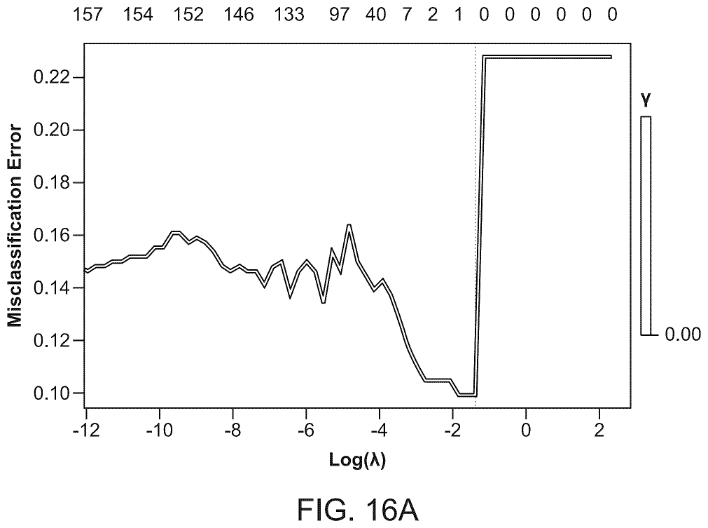

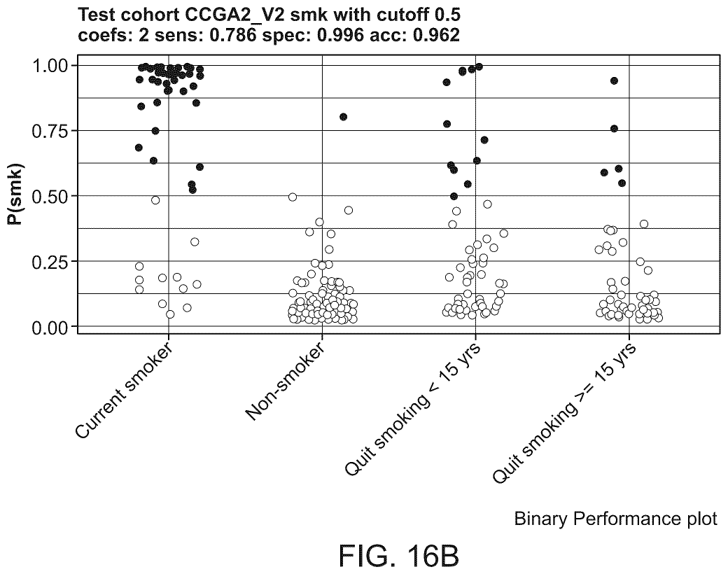

FIG. 16A illustrates identification of a feature set of genomic regions for predicting smoking status, in accordance with one or more example implementations. The training samples utilized were the CCGA2 study. The analytics system performs a glmnet relaxed lasso regression on a lambda grid to identify an optimal range of genomic regions to use as the feature set. The optimal set of genomic regions in the range of 1 to 4 provided the lowest mean-squared error (plotted on the y-axis). A feature set of 2 genomic regions were used to train the smoking status prediction model for the test sets.

FIG. 16B illustrates results from a trained smoking status sex prediction model, in accordance with example implementations. The smoking status prediction model was trained on the 2 genomic regions identified in FIG. 16A. The test cohort comprise non-cancer samples. In sum, the smoking status prediction model was 96.2% accurate with 99.6% specificity.

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method comprising:
obtaining a plurality of training samples, each training sample:
comprising a plurality of nucleic acid fragments, each of the plurality of nucleic acid fragments having a genomic location overlapping at least one genomic region of a plurality of genomic regions, and
labelled with a chronological age of an individual from whom the training sample is derived;
sequencing the plurality of nucleic acid fragments for each training sample to identify a methylation pattern for each nucleic acid fragment;
for each genomic region of a plurality of genomic regions,
identifying nucleic acid fragments from the plurality having genomic locations overlapping the genomic region, and
calculating, for the genomic region, an indicativeness score representing a correlation between chronological age and methylation patterns, and calculated based on chronological ages of individuals from whom the identified nucleic acid fragments are derived and methylation patterns of identified nucleic acid fragments;
generating a feature set comprising one or more genomic regions of the plurality of genomic regions, the one or more genomic regions in the feature set having indicativeness scores above a threshold; and
training a machine-learned age-prediction model to determine a predicted chronological age of a tested individual from whom a test sample is derived, the training based on methylation patterns of nucleic acid fragments in the plurality of training samples overlapping the one or more genomic regions in the feature set.

2. The method of claim 1, further comprising:
training a linear regression for each genomic region of the feature set based on the methylation patterns of the nucleic acid fragments overlapping each genomic region from training samples of the plurality labelled as non-cancer;
obtaining a plurality of additional training samples, each additional training sample:
comprising a plurality of additional nucleic acid fragments having additional genomic locations overlapping at least one genomic region of the plurality of genomic regions,
labelled with a chronological age of an individual from whom the additional training sample was derived, and
labelled as non-cancer or cancer based on a previous determination of cancer presence in the additional training sample;
sequencing the plurality of additional nucleic acid fragments to identify a methylation pattern for each additional nucleic acid fragment;
for each genomic region of the plurality:
applying the linear regression to methylation patterns of nucleic acid fragments of the plurality of additional training samples to determine a predicted chronological age of the individual from whom the additional training sample was derived,
calculating age residuals for each additional training sample as a difference between its predicted chronological age and its labelled chronological age, and
comparing age residuals of the additional training samples labelled as cancer to age residuals of the additional training samples labelled as non-cancer; and
generating a reduced feature set from the feature set based on the comparison of age residuals, wherein the reduced feature set comprises a lesser number of genomic regions than the feature set, and the reduced feature set is used to train the machine-learned age-prediction model.

3. The method of claim 1, further comprising:
obtaining a test sample, the test sample comprising a plurality of additional nucleic acid fragments and labelled with a chronological age of a test subject from whom the test sample is derived;
sequencing the plurality of additional nucleic acid fragments for the test sample to identify methylation patterns for the additional nucleic acid fragments of the plurality;
applying the trained age-prediction model to determine a predicted chronological age of the test subject from whom the test sample was derived based on methylation patterns of the additional nucleic acid fragments overlapping the one or more genomic regions in the feature set;
calculating an age residual as a difference between the labelled chronological age and the predicted chronological age of the test subject; and
determining that the test sample has a strong likelihood for presence of cancer in response to determining that the age residual is above a residual threshold.

4. The method of claim 3, wherein the residual threshold is determined by:
applying the trained age-prediction model to a second plurality of training samples identified as non-cancer to determine a predicted age for each of the second plurality of training samples;
calculating an age residual for each of the second plurality of training samples by comparing the predicted age to a labelled chronological age of the second plurality of training samples; and
identifying the residual threshold based on the calculated age residuals for the second plurality of training samples, wherein at least a majority of the calculated age residuals for the second plurality of training samples satisfy the residual threshold.

5. The method of claim 3, further comprising:
in response to determining that the test sample has the strong likelihood for presence of cancer:
filtering the methylation patterns of the plurality of additional nucleic acid fragments with p-value filtering to identify a set of anomalous methylation patterns;
generating a feature vector for the test sample based on the age residual and the set of anomalous methylation patterns; and
determining a cancer prediction for the test sample by inputting the feature vector into a trained cancer classifier.

6. The method of claim 5, wherein the cancer prediction is a binary prediction between presence and absence of cancer or another disease state.

7. The method of claim 5, wherein the cancer prediction is a multiclass prediction between a plurality of cancer types.

8. The method of claim 5, wherein the cancer prediction is a multiclass prediction between a plurality of disease states.

9. The method of claim 3, further comprising:
determining a presence of cancer in the test sample using a secondary machine-learned cancer classifier, the secondary cancer classifier configured to receive as input the predicted chronological age of the subject and methylation patterns of the plurality of additional nucleic acid fragments and output a prediction of the presence of cancer in the test sample.

10. The method of claim 9, wherein the secondary machine-learned cancer classifier is further configured to receive as input clinical information and genetic background of the subject and output the prediction of the presence of cancer in the test sample.

11. (canceled)

12. (canceled)

13. The method of claim 1, wherein the indicativeness score is determined by training a linear regression to regress chronological age from methylation density of non-cancer training samples, wherein methylation density is calculated as a percentage of nucleic acid fragments having genomic locations which overlap a particular genomic region having a methylated state in that particular genomic region.

14. The method of claim 1, wherein the machine-learned age-prediction model comprises a multivariate regression.

15. (canceled)

16. The method of claim 14, wherein the machine-learned age-prediction model receives as input a methylation density corresponding to each of the genomic regions in the feature set.

17. The method of claim 1, wherein a number of the one or more genomic regions in the feature set is selected from a range of 5-10,000.

18. The method of claim 1, wherein sequencing the nucleic acid fragments comprises whole genome bisulfite sequencing (WGBS).

19. The method of claim 1, wherein sequencing the nucleic acid fragments comprises targeted sequencing.

20. (canceled)

21. (canceled)

22. (canceled)

23. The method of claim 1, wherein each training sample is labeled with a sex or a smoking status of the individual from whom the training sample is derived, and comprising:
calculating, for the genomic region, an additional indicativeness score representing a correlation between sex or smoking status and methylation patterns, and
training a machine-learned characteristic prediction model to determine a predicted sex or smoking status of a tested individual from whom a test sample is derived.

24. (canceled)

25. (canceled)

26. (canceled)

27. (canceled)

28. (canceled)

29. (canceled)

30. (canceled)

31. (canceled)

32. A non-transitory computer readable storage medium comprising computer program instructions that, when executed by one or more processors, cause the one or more processors to:
obtain a plurality of training samples, each training sample:
comprising a plurality of nucleic acid fragments, each of the plurality of nucleic acid fragments having a genomic location overlapping at least one genomic region of a plurality of genomic regions, and
labelled with a chronological age of an individual from whom the training sample is derived;
sequence the plurality of nucleic acid fragments for each training sample to identify a methylation pattern for each nucleic acid fragment;
for each genomic region of a plurality of genomic regions,
identify nucleic acid fragments from the plurality having genomic locations overlapping the genomic region, and
calculate, for the genomic region, an indicativeness score representing a correlation between chronological age and methylation patterns, and calculated based on chronological ages of individuals from whom the identified nucleic acid fragments are derived and methylation patterns of identified nucleic acid fragments;
generate a feature set comprising one or more genomic regions of the plurality of genomic regions, the one or more genomic regions in the feature set having indicativeness scores above a threshold; and
train a machine-learned age-prediction model to determine a predicted chronological age of a tested individual from whom a test sample is derived, the training based on methylation patterns of nucleic acid fragments in the plurality of training samples overlapping the one or more genomic regions in the feature set.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A system comprising:
one or more processors;
a non-transitory computer readable storage medium storing computer program instructions that, when executed by the one or more processors, cause the one or more processors to:
obtain a plurality of training samples, each training sample:
comprising a plurality of nucleic acid fragments, each of the plurality of nucleic acid fragments having a genomic location overlapping at least one genomic region of a plurality of genomic regions, and
labelled with a chronological age of an individual from whom the training sample is derived;
sequence the plurality of nucleic acid fragments for each training sample to identify a methylation pattern for each nucleic acid fragment;
for each genomic region of a plurality of genomic regions,
identify nucleic acid fragments from the plurality having genomic locations overlapping the genomic region, and
calculate, for the genomic region, an indicativeness score representing a correlation between chronological age and methylation patterns, and calculated based on chronological ages of individuals from whom the identified nucleic acid fragments are derived and methylation patterns of identified nucleic acid fragments;
generate a feature set comprising one or more genomic regions of the plurality of genomic regions, the one or more genomic regions in the feature set having indicativeness scores above a threshold; and
train a machine-learned age-prediction model to determine a predicted chronological age of a tested individual from whom a test sample is derived, the training based on methylation patterns of nucleic acid fragments in the plurality of training samples overlapping the one or more genomic regions in the feature set.

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)
